(19) United States
(12) Patent Application Publication
(10) Pub. No.: US 2025/0194905 A1
(43) Pub. Date: Jun. 19, 2025

(54) MODULAR DISTAL ASSEMBLIES FOR MEDICAL DEVICES AND METHODS OF USING AND MAKING THE SAME

(71) Applicant: Boston Scientific Scimed, Inc., Maple Grove, MN (US)

(72) Inventors: Nathan GAWORSKI, St. Paul, MN (US); Daniel J. FOSTER, Lino Lakes, MN (US); Kirsten VIERING, Newton, MA (US); Bradley S. SWEHLA, Eagan, MN (US); Craig Michael WILSON, Minneapolis, MN (US)

(73) Assignee: Boston Scientific Scimed, Inc., Maple Grove, MN (US)

(21) Appl. No.: 18/985,204

(22) Filed: Dec. 18, 2024

Related U.S. Application Data
(60) Provisional application No. 63/611,855, filed on Dec. 19, 2023.

(51) Int. Cl. A61B 1/00; A61B 1/06

(52) U.S. Cl. CPC A61B 1/00137; A61B 1/0011; A61B 1/0653

(57) ABSTRACT

A medical device that includes a handle, a shaft extending from the handle, and an assembly coupled to a distal end of the shaft. The assembly includes an end cap including first and second channels, an imaging mechanism coupled to the end cap and received within the first channel, and a lighting mechanism coupled to the end cap and received within the second channel. The assembly includes a first circuit assembly coupled to the end cap and a second circuit assembly coupled to the first circuit assembly. The imaging and lighting mechanisms are mounted onto the first circuit assembly. The first circuit assembly includes a rigid circuit board communicatively coupled to the imaging and lighting mechanisms. The second circuit assembly includes a flexible circuit board interposed between and communicatively coupled to the first circuit assembly and a control system of the medical device.

### CROSS-REFERENCE TO RELATED APPLICATIONS

This application claims the benefit of priority of U.S. Provisional Patent Application No. 63/611,855, filed on Dec. 19, 2023, the entirety of which is incorporated herein by reference.

### TECHNICAL FIELD

Various aspects of this disclosure relate generally to modular medical systems, devices, and related methods. For example, the disclosure includes systems, devices, and related methods for manufacturing and assembling a medical device including a modular tip assembly that integrates multiple components.

### BACKGROUND

In certain medical procedures, it may be necessary to utilize a medical device that incorporates advanced and differentiating features to treat a procedure site within a subject (e.g., a patient). For example, an endoscopic medical procedure may require the use of imaging, lighting, pressure sensing, or other features to facilitate access and treatment of the procedure site, such as in the gastrointestinal tract of the subject, for example in the esophagus, stomach, or intestines. During an endoscopic procedure, a user inserts a sheath of an endoscope into a body lumen of the subject. The user utilizes a handle of the endoscope to control the endoscope during the procedure. Tools may be passed through a working channel of the endoscope via, for example, a port in the handle, to deliver treatment at the procedure site near a distal end of the endoscope, which is remote from the user.

To access, visualize, and navigate to the procedure site, however, numerous devices and complex assemblies may be necessary for use with the endoscope, either as instruments passed through the working channel or manufactured as a component of the endoscope. Use of the endoscope with additional instruments may complicate the medical procedure, while manufacturing the endoscope to include the instruments may require use of various manufacturing methods and dedicated manufacturing assembly lines, thereby increasing costs. Aspects of this disclosure may help to solve one or more of these issues or other issues in the art.

### SUMMARY

Aspects of the disclosure relate to, among other things, systems, devices, and methods for a medical device including a modular tip assembly with mechanisms for illuminating, imaging, sensing, and accessing a procedure site, among other aspects. Each of the aspects disclosed herein may include one or more of the features described in connection with any of the other disclosed aspects.

According an example, a medical device includes a handle; a shaft extending distally from the handle; and an assembly coupled to a distal end of the shaft, wherein the assembly includes: an end cap including a first channel and a second channel; an imaging mechanism coupled to the end cap and received within the first channel; a lighting mechanism coupled to the end cap and received within the second channel; a first circuit assembly coupled to the end cap, wherein the imaging mechanism and the lighting mechanism are mounted onto the first circuit assembly; and a second circuit assembly coupled to the first circuit assembly, the second circuit assembly is flexibly deformable to selectively position the first circuit assembly in alignment with the end cap; wherein the end cap includes a material received within the second channel and disposed over the lighting mechanism, the material being configured to modify a light emitted outwards from the end cap by the lighting mechanism; and wherein the first circuit assembly includes a rigid circuit board communicatively coupled to the imaging mechanism and the lighting mechanism, and the second circuit assembly includes a flexible circuit board interposed between and communicatively coupled to the first circuit assembly and a control system of the medical device.

Any of the medical devices described herein may include any of the following features. The end cap includes a port positioned adjacent to and in fluid communication with the first channel, wherein the port is configured to receive a second material that extends into the first channel to securely attach the imaging mechanism to the first channel. The end cap includes a plurality of divots positioned about and in fluid communication with the first channel, the plurality of divots defining a gap that extends about a perimeter of the imaging mechanism within the first channel. The gap is configured to receive the second material from the port, thereby encapsulating the imaging mechanism with the second material. The assembly includes a sensor configured to detect a pressure applied to the distal end of the shaft. The end cap is formed of a thermally conductive material that is configured to dissipate heat generated by the lighting mechanism and the imaging mechanism. The flexible circuit board includes a first segment coupled to a wire assembly that is communicatively coupled to the control system, a second segment coupled to the rigid circuit board, and a third segment positioned between the first segment and the second segment. The first segment, the second segment, and the third segment are individually movable relative to one another to arrange the wire assembly and the rigid circuit board at predefined configurations relative to one another. The material includes a first material, and the end cap includes a second material received within the second channel and disposed between the first material and the lighting mechanism, such that the second material is configured to separate the first material from the lighting mechanism. The end cap includes a third material received within the second channel and disposed over the first material, the third material is configured to evenly diffuse the light emitted by the lighting mechanism and modified by the first material when exiting the first channel. The first material includes a phosphor layer, the second material includes a silicone layer, and the third material includes a diffuser layer. The lighting mechanism includes a light-emitting diode that is configured to emit blue light, and the material includes a phosphor formulation that is configured to modify the blue light to white light. The shaft includes an articulation segment at the distal end of the shaft that is configured to move the assembly relative to the shaft. The assembly includes a sleeve coupled between the end cap and the articulation segment, the sleeve is configured to house the first circuit assembly and the second circuit assembly; wherein the sleeve includes an aspiration port that is in fluid communication with an aspiration channel of the shaft.

According to another example, a medical device includes a handle; a shaft extending distally from the handle; and an assembly coupled to a distal end of the shaft, wherein the assembly includes: an end cap; a pair of first circuit assemblies coupled to the end cap, wherein each of the pair of first circuit assemblies includes an imaging mechanism and a lighting mechanism mounted onto the first circuit assembly; and a second circuit assembly coupled to each of the pair of first circuit assemblies, wherein the second circuit assembly is flexibly deformable to selectively position the pair of first circuit assemblies in predefined configurations relative to the end cap; wherein each of the pair of first circuit assemblies includes a material disposed over the lighting mechanism, the material being configured to modify a light emitted outwards from the end cap by each lighting mechanism from the pair of first circuit assemblies; and wherein the pair of first circuit assemblies include rigid circuit boards and the second circuit assembly includes a flexible circuit board.

Any of the medical devices described herein may include any of the following features. Each of the pair of first circuit assemblies includes a channel configured to receive the imaging mechanism and a port in fluid communication with the channel, the port is configured to guide a second material into the channel to securely attach the imaging mechanism to the channel; wherein each of the pair of first circuit assemblies includes a plurality of divots in fluid communication with the channel and defining a gap about a perimeter of the imaging mechanism within the channel, the gap is configured to receive the second material from the port, thereby encapsulating the imaging mechanism with the second material. Each of the pair of first circuit assemblies includes a rigid circuit board communicatively coupled to the imaging mechanism and the lighting mechanism, and the second circuit assembly includes a flexible circuit board interposed between and communicatively coupled to the pair of first circuit assemblies and a control system of the medical device. The material includes a first material, and the end cap includes a second material disposed between the first material and the lighting mechanism to separate the first material from the lighting mechanism, and a third material disposed over the first material that is configured to evenly diffuse the light emitted by the lighting mechanism and modified by the first material upon exiting the end cap. The first material includes a phosphor layer, the second material includes a silicone layer, and the third material includes a diffuser layer.

According to another example, a method of assembling a plurality of medical devices includes coupling a first circuit assembly onto each of a plurality of panelized modules of a panel including the plurality of panelized modules, wherein the panel is formed of a flexible circuit board such that each of the plurality of panelized modules defines a second circuit assembly; mounting an imaging mechanism and a lighting mechanism onto each of the first circuit assemblies that are coupled to the plurality of panelized modules; attaching an end cap over each of the first circuit assemblies that include the imaging mechanism and the lighting mechanism mounted thereon, the end cap is configured to house the imaging mechanism and the lighting mechanism; filling the end cap with one or more materials to encapsulate the imaging mechanism and the lighting mechanism within the end cap; and detaching the plurality of panelized modules from the panel to form a plurality of modular tip assemblies for attachment to a shaft of the plurality of medical devices.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of an exemplary medical device including a modular tip assembly, according to aspects of this disclosure;

FIG. 2 is a partially transparent view of the modular tip assembly of the medical device of FIG. 1, according to aspects of this disclosure;

FIG. 3 is an exploded view of the modular tip assembly of FIG. 2, according to aspects of this disclosure;

FIG. 4 is a cross-sectional view of the modular tip assembly of FIG. 2 taken along line 4-4 of FIG. 2, according to aspects of this disclosure;

FIGS. 5-6 are partial perspective views of the modular tip assembly of FIG. 2, according to aspects of this disclosure;

FIGS. 7-8 are perspective views of a method of assembling the modular tip assembly of FIG. 2 on a panel, according to aspects of this disclosure;

FIG. 9 is a perspective view of another modular tip assembly of the medical device of FIG. 1, according to aspects of this disclosure;

FIG. 10 is a partially transparent view of the modular tip assembly of FIG. 9, according to aspects of this disclosure;

FIG. 11 is a perspective view of the modular tip assembly of FIG. 9, according to aspects of this disclosure;

FIG. 12 is an exploded view of the modular tip assembly of FIG. 9, according to aspects of this disclosure;

FIG. 13 is a partial perspective view of the modular tip assembly of FIG. 9, according to aspects of this disclosure;

FIG. 14 is a perspective view of another modular tip assembly of the medical device of FIG. 1, according to aspects of this disclosure;

FIG. 15 is a partial perspective view of the modular tip assembly of FIG. 14, according to aspects of this disclosure;

FIG. 16 is a partial side view of the modular tip assembly of FIG. 14, according to aspects of this disclosure;

FIGS. 17-19 are perspective views of a method of assembling the modular tip assembly of FIG. 14, according to aspects of this disclosure;

FIG. 20 is a perspective view of another modular tip assembly of the medical device of FIG. 1, according to aspects of this disclosure;

FIG. 21 is a partial perspective view of the modular tip assembly of FIG. 20, according to aspects of this disclosure;

FIG. 22 is a front elevational view of the modular tip assembly of FIG. 20, according to aspects of this disclosure; and

FIG. 23 is a schematic view of a method of assembling the modular tip assembly of FIG. 20 on a panel, according to aspects of this disclosure.

### DETAILED DESCRIPTION

This disclosure relates, in certain aspects, to medical devices with a modular tip assembly including customizable components and/or mechanisms for facilitating treatment of a procedure site in a subject (e.g., patient). For example, medical devices may be equipped with a modular tip assembly that integrates a customized combination of components, devices, and/or mechanisms that are arranged relative to one another in an arrangement that is particularly configured and operable to facilitate accessing, positioning, and treating the procedure site. The tip assembly may be modular in that different tip assemblies with varying configurations may be assembled onto the same medical device. In some procedures, a certain configuration of the modular tip assembly may enhance the capabilities of the medical device in treating the procedure site by positioning the components, devices, and/or mechanisms at particular relative arrangements, such as arrangements based on the procedure or location of the procedure site within the subject.

In examples, accessing a procedure site may include endoluminal placement of the medical device into the patient, such as through an anatomical passageway via a natural orifice. The orifice can be, for example, the nose, mouth, or anus, and the placement can be in any portion of the GI tract, including the esophagus, stomach, duodenum, large intestine, or small intestine. Placement also can be in other organs or other bodily spaces reachable via the GI tract, other body lumens, or openings in the body. This disclosure is not limited to any particular medical procedure or treatment site within a body.

Examples of the disclosure may relate to devices and methods for performing various medical procedures and/or treating portions of the large intestine (colon), small intestine, cecum, esophagus, pancreatic-biliary tract, any other portion of the gastrointestinal tract, and/or any other suitable patient anatomy (collectively referred to herein as a “target treatment site”). As mentioned above, this disclosure is not limited to any specific medical device or method, and aspects of the disclosure may be used in connection with any suitable medical tool and/or medical method, at any suitable site within the body.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

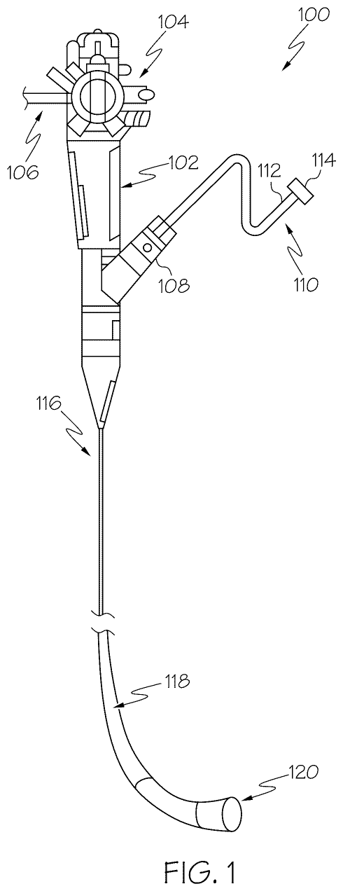

FIG. 1 depicts an exemplary medical device 100. Medical device 100 may include an insertion device, such as an endoscope, which may be inserted into an esophagus of a patient. Medical device 100 may include a handle 102 and a shaft 116 extending distally from handle 102. Shaft 116 may include one or more channels extending therethrough from a proximal portion positioned adjacent to handle 102 to a distal portion 118 terminating at a modular assembly 120. In some embodiments, medical device 100 may include an umbilicus (not shown), which may connect a first port 106 on handle 102 to sources of, for example, air, water, suction, power, image processing and/or viewing equipment. In some embodiments, medical device 100 may include an imaging element and/or a lighting element, such as at modular assembly 120, to aid in accurately positioning shaft 116 adjacent a procedure site during a procedure.

Using one or more of the channels of shaft 116, such as a working channel, a user of medical device 100 may deploy or otherwise deliver a medical tool or instrument to the target treatment site, such as a medical instrument 110 received through a second port 108 on handle 102. For example, medical instrument 110 may include a shaft 112 that accesses and extends through the working channel of shaft 116 via second port 108, and a proximal end 114 that is disposed external to handle 102. Proximal end 114 may define a user interface of medical instrument 110 for access and control by a user of medical instrument 110. As described herein, modular assembly 120 may include one or more openings facilitating access from or to the working channel of shaft 116 such that a distal end of medical instrument 110 (not shown) may extend out of shaft 116 at modular assembly 120 for accessing the procedure site when distal portion 118 is positioned at the procedure site.

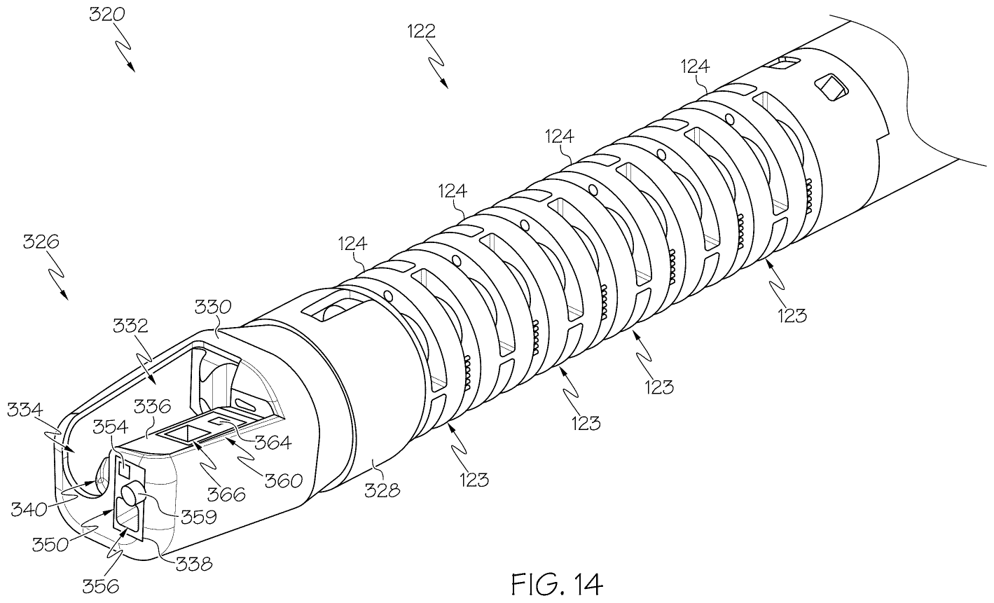

Still referring to FIG. 1, handle 102 may include one or more actuators 104 at or along a proximal end of handle 102 (e.g., adjacent to port 106), for example, to control the movement of shaft 116, and particularly distal portion 118, the activation of one or more imaging element(s) and lighting element(s), and control a deflection, position, or orientation of modular assembly 120. It should be appreciated that control of modular assembly 120 may be further provided by moving handle 102, such as by pushing and/or pulling handle 102 to advance or retract modular assembly 120 relative to the procedure site. It is noted that FIG. 1 illustrates modular assembly 120 of medical device 100 (e.g., an endoscope) as being “forward-facing” in that the features of modular assembly 120, such as the one or more channels of shaft 116 and mechanisms of modular assembly 120, may face distally (i.e., forward of a distalmost face of modular assembly 120). It should be appreciated that this disclosure also encompasses other configurations of a modular assembly, including modular assemblies that are “side-facing” in which the one or more channels of shaft 116 and mechanisms of the modular assembly may be disposed on a radially outer side of the modular assembly (see FIGS. 14-19). In this instance, said channels and mechanisms may be positioned in a radially outward direction, approximately perpendicularly to a longitudinal axis of distal portion 118.

Although medical device 100 is discussed above as being an endoscope, this disclosure is not so limited. Although the disclosure may refer at different points to an endoscope, it will be appreciated that, unless otherwise specified, duodenoscopes, endoscopes, gastroscopes, endoscopic ultrasonography (“EUS”) scopes, colonoscopes, ureteroscopes, bronchoscopes, laparoscopes, cytoscopes, aspiration scopes, sheaths, catheters, or any other suitable delivery device or insertion device may be used in connection with the systems, devices, elements, assemblies, methods, etc. described herein.

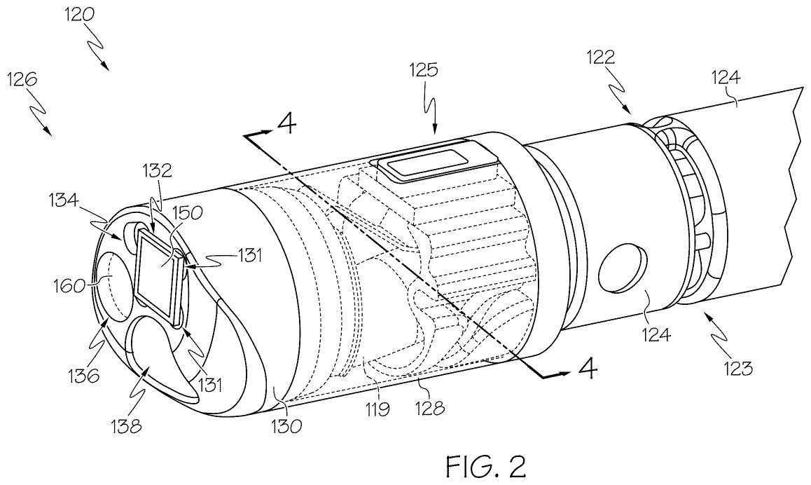

Referring now to FIG. 2, modular assembly 120 may include an articulation (proximal) segment 122 and a tip (distal) segment 126, positioned distally relative to articulation segment 122. In the example, tip segment 126 may be coupled to a terminal (distalmost) end of articulation segment 122. Articulation segment 122 may include a plurality of links 124 that are separated from one another by a cutout 123. The plurality of links 124 may be configured to move relative to one another due to the corresponding cutouts 123 positioned therebetween, thereby allowing movement of articulation segment 122 relative to distal portion 118 (see FIG. 1). Any alternative type of articulation segment (e.g., articulation joint) may be used in place of articulation segment 122.

With tip segment 126 positioned at the terminal (distal) end of articulation segment 122, articulation segment 122 may be configured to articulate and/or deflect tip segment 126 towards a plurality of directions and/or positions. In some embodiments, a distal portion of articulation segment 122 may be configured to move laterally relative to distal portion 118 (FIG. 1), thereby deflecting tip segment 126 in a corresponding lateral direction. In other embodiments, articulation segment 122 may be configured to move in various other suitable directions relative to distal portion 118, thereby deflecting tip segment 126 accordingly. In some embodiments, articulation segment 122 may be joint molded, while in other embodiments articulation segment 122 may be manufactured via various other suitable methods.

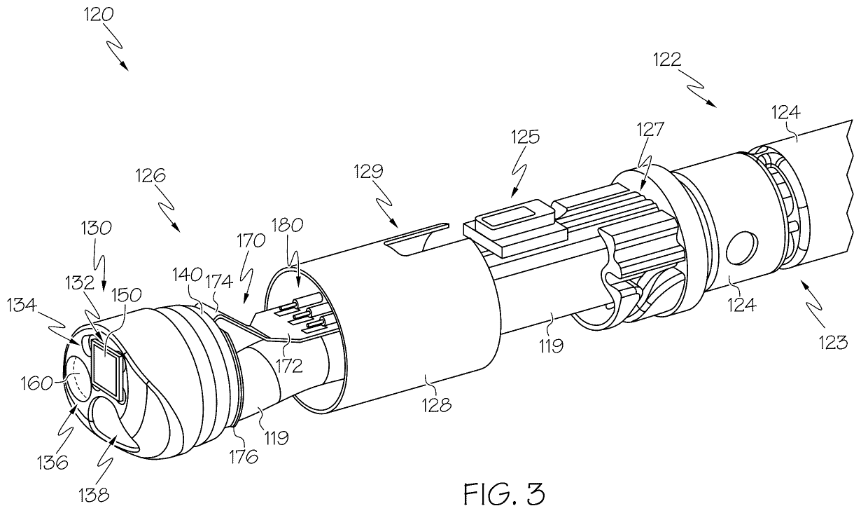

Tip segment 126 of modular assembly 120 may include a sleeve 128 and an end cap 130 that are collectively sized, shaped, and/or otherwise configured to house one or more components of modular assembly 120. In the example, sleeve 128 may be coupled to the terminal (distalmost) end of articulation segment 122, and end cap 130 may be coupled to sleeve 128 along an opposite end from articulation segment 122. For example, end cap 130 may be coupled to a distal end of sleeve 128. Modular assembly 120 may include a sensor 125 at least partially disposed within sleeve 128. In the example, sleeve 128 may include an opening 129 (see FIG. 3) along an exterior surface of sleeve 128 that is sized, shaped, and/or otherwise configured to receive sensor 125. In this instance, sensor 125 may be configured to detect one or more parameters from the exterior of sleeve 128. In some embodiments, sensor 125 may include a pressure sensing device that is operable to detect a pressure applied to modular assembly 120. In other embodiments, sensor 125 may include various other suitable sensing devices without departing from a scope of this disclosure, including but not limited to, sensors operable for temperature or electromagnetic tracking. In other embodiments, modular assembly 120 may omit sensor 125 entirely.

Still referring to FIG. 2, end cap 130 may include a plurality of channels that open at a distal face of end cap 130. As described herein, one or more mechanisms of modular assembly 120 may be received within the plurality of channels of end cap 130. In this instance, modular assembly 120 may be generally distal and/or forward facing. For example, end cap 130 may include a first channel 132, a second channel 136, and a third channel 138 that open at the distal face of end cap 130. In other embodiments, end cap 130 may include one or more channels that open at various other faces of end cap 130 than the distal face, such as a lateral (side) face of end cap 130, without departing from a scope of this disclosure. In this instance, modular assembly 120 may be lateral and/or side facing.

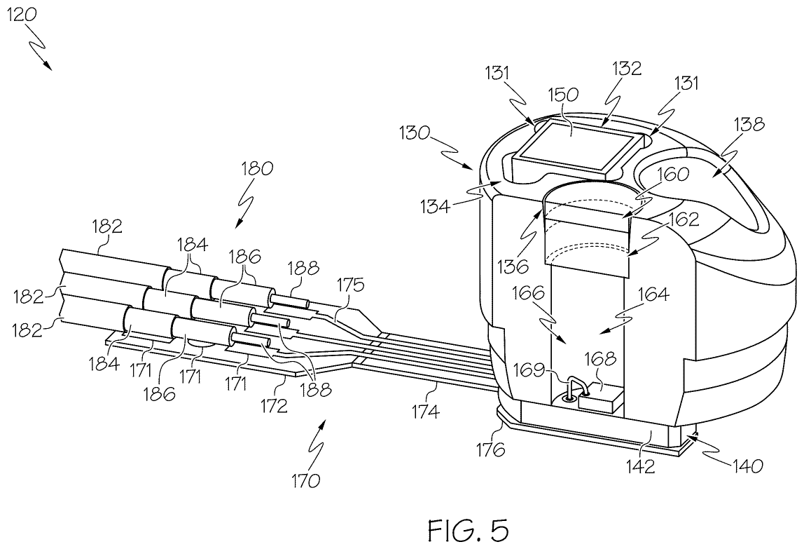

In the example, first channel 132 may be sized, shaped, and/or otherwise configured to receive an imaging mechanism 150 (e.g., a camera or other component with an image sensor). Second channel 136 may be sized, shaped, and/or otherwise configured to receive a lighting mechanism 168 (see FIG. 5), such as one or more light emitting diode(s) or optical fiber(s). Third channel 138 may be sized, shaped, and/or otherwise configured to facilitate access to and/or from a working channel 119 of shaft 116 for receiving one or more instruments (e.g., medical instrument 110 of FIG. 1) extending through shaft 116 (FIG. 1). Stated differently, third channel 138 may be fluidly coupled to working channel 119.

Still referring to FIG. 2, end cap 130 may include one or more divots 131 (e.g., cutouts) that at least partially define an opening of first channel 132. In other words, the one or more divots 131 may collectively define and/or increase a cross-sectional profile of first channel 132. In the example, first channel 132 may be generally squared and end cap 130 may include at least one divot 131 at each edge and/or corner of the squared opening of first channel 132. As described herein, divots 131 may be configured and operable to facilitate encapsulation of imaging mechanism 150 within first channel 132 by a material during assembly of modular assembly 120. End cap 130 may further include a fill port 134 positioned along at least one of the sides, corners, and/or edges of first channel 132, such that fill port 134 may be in fluid communication with first channel 132.

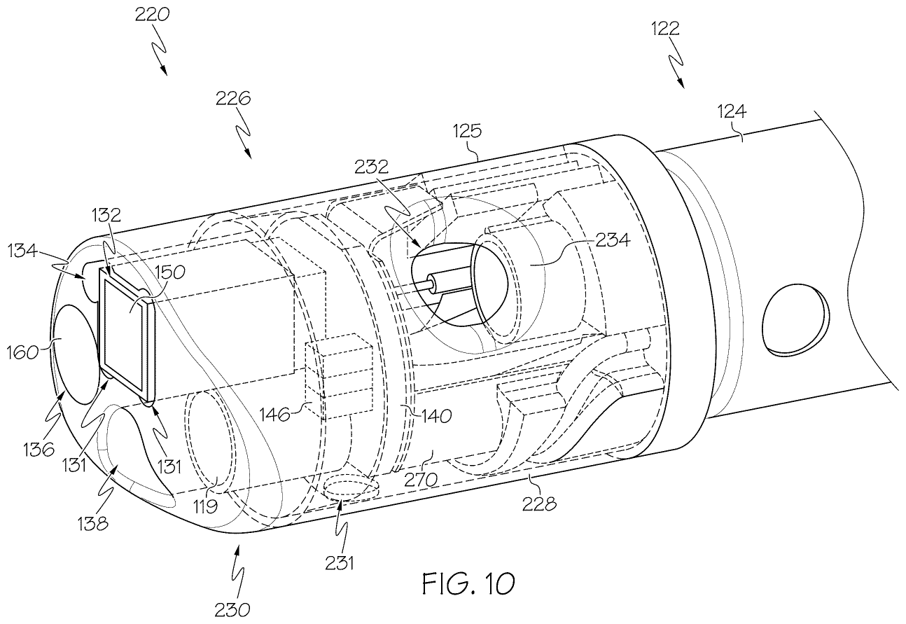

Fill port 134 may be sized, shaped, and/or otherwise configured to receive and guide a material (e.g., an adhesive or any type of encapsulating material) into first channel 132 to securely couple imaging mechanism 150 to end cap 130. The one or more divots 131 may form a gap extending about an exterior perimeter of imaging mechanism 150 when imaging mechanism 150 is received within first channel 132, and the material (e.g., an adhesive) received within first channel 132 via fill port 134 may extend about an exterior of imaging mechanism 150 and into the gaps formed by divots 131, thereby securing imaging mechanism 150 to end cap 130. In some embodiments, the material (e.g., adhesive) received within first channel 132 via fill port 134 may extend through end cap 130 and into sleeve 128 and/or other portions of tip segment 126 to securely attach additional components and/or assemblies of modular assembly 120 to one another. Additionally and/or alternatively, the material received via fill port 134 may be operable to waterproof interior cavities of tip segment 126. Although not shown, in other embodiments sleeve 128 may include one or more fill ports for facilitating receipt of additional material (e.g., adhesive or any type of encapsulating material) to encapsulate the internal components and/or assemblies of modular assembly 120 (see FIG. 10).

Still referring to FIG. 2, end cap 130 may include an outer cover 160 disposed at an opening of second channel 136, thereby enclosing the lighting mechanism 168 (FIG. 5) within second channel 136. In some embodiments, outer cover 160 may be coupled to end cap 130 at the opening of second channel 136. As described in detail herein, outer cover 160 may include a material that is configured and operable to diffuse light emitted by lighting mechanism 168 from within second channel 136. In other words, outer cover 160 may include a light diffuser that is configured and operable to evenly diffuse the light emitted by lighting mechanism 168. In some embodiments, lighting mechanism 168 may be communicatively coupled to a plastic optical fiber (POF) extending through shaft 116 of medical device 100, with the POF being configured and operable to transmit light to lighting mechanism 168.

As best seen in FIG. 3, sensor 125 may be communicatively coupled to a control system of medical device 100 (not shown) via a wired connection, such as through one or more wires 127 that are electrically coupled to a control system (not shown) of medical device 100 that is operable to control operation of the one or more systems, devices, and/or mechanisms of modular assembly 120. In some embodiments, the control system may include a circuit board disposed within handle 102, which may be in communication with the umbilicus (not shown) connected to medical device 100 via first port 106. In other embodiments, sensor 125 may be communicatively coupled to the control system via a wireless connection, such that wires 127 may be omitted entirely.

Modular assembly 120 may include a first circuit assembly 140 and a second circuit assembly 170, each of which may be disposed at least partially within sleeve 128 and/or end cap 130. In the example, first circuit assembly 140 may include a rigid circuit board, and second circuit assembly 170 may include a flexible circuit board that is flexibly deformable. As described herein, the rigid circuit board of first circuit assembly 140 may be mounted to the flexible circuit board of second circuit assembly 170, and the flexible circuit board may be flexibly deformable to selectively position the rigid circuit board in alignment with the channels of end cap 130. In this instance, second circuit assembly 170 may be configured to orient the internal mechanisms in modular assembly 120 to be bent perpendicularly relative to shaft 116, thereby providing a distal-facing modular assembly 120 on medical device 100.

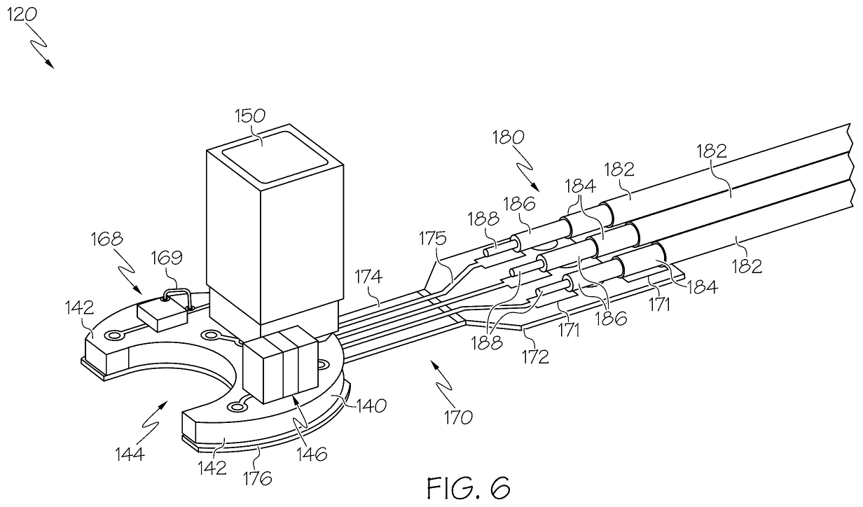

First circuit assembly 140 may be communicatively coupled to the one or more mechanisms disposed within end cap 130, such as imaging mechanism 150 and lighting mechanism 168 (see FIG. 5). As shown and described in detail herein, first circuit assembly 140 may include a body 142 that is generally U-shaped, C-shaped, and/or horseshoe shaped (see FIG. 6). Body 142 may be sized, shaped, and/or otherwise configured to facilitate receipt of imaging mechanism 150 and lighting mechanism 168 thereon. Body 142 may define a gap 144 that may be configured to align with third channel 138 when first circuit assembly 140 is coupled to end cap 130, thereby allowing medical instrument 110 from working channel 119 to pass through gap 144 and into third channel 138. In some examples, gap 144 may have an edge that is arcuate shaped (e.g., shaped like a portion of a circumference of a circle). First circuit assembly 140 may be coupled to end cap 130 along a first (distal) face of first circuit assembly 140, and coupled to second circuit assembly 170 along a second (proximal) face of first circuit assembly 140 that is opposite of the first (distal) face.

Still referring to FIG. 3, second circuit assembly 170 may include a plurality of segments, such as but not limited to, a first (proximal) segment 172, a second (intermediate) segment 174 that is positioned distally relative to first segment 172, and a third (distal) segment 176 that is positioned distally relative to second segment 174 and first segment 172. In some embodiments, each of first segment 172, second segment 174, and third segment 176 may be integral with one another, such that the plurality of segments of second circuit assembly 170 may have a unitary body. In other embodiments, one or more of first segment 172, second segment 174, and/or third segment 176 may separate pieces that are coupled and/or attached to one another.

In the example, first segment 172 may have a longitudinal length that extends in a substantially parallel direction relative to a longitudinal axis of modular assembly 120, with upper and lower surfaces of first segment 172 oriented to face opposing directions that are transverse to (e.g., perpendicular to) the longitudinal axis of modular assembly 120. Second segment 174 may have a longitudinal length that extends in a transverse direction relative to the longitudinal axis of modular assembly 120. In this instance, second segment 174 may have an angled configuration (a non-zero angle) relative to first segment 172 with upper and lower surfaces of second segment 174 oriented to face opposing directions that are transverse to the longitudinal axis of modular assembly 120. Third segment 176 may have a longitudinal length that extends in a substantially transverse direction (e.g., perpendicular direction) relative to the longitudinal axis of modular assembly 120, such that front and rear surfaces of third segment 176 may be oriented to face opposing directions that are parallel to the longitudinal axis of modular assembly 120.

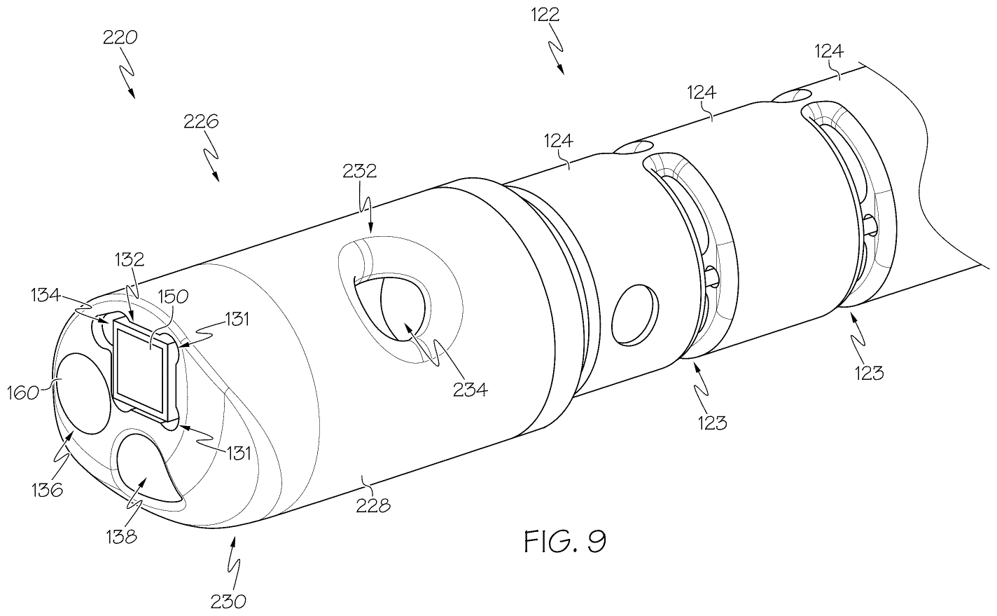

It should be appreciated that a relative configuration of second circuit assembly 170, and particularly the arrangement of first segment 172, second segment 174, and/or third segment 176 relative to one another, may be selectively adjusted to various other suitable positions based on a modular configuration of modular assembly 120 (see FIGS. 9-21). As such, one or more of the segments of second circuit assembly 170 may be individually movable relative to one another to arrange modular assembly 120 at a predefined configuration during assembly. Additionally, second circuit assembly 170 may include additional and/or fewer segments than those shown and described herein without departing from a scope of this disclosure.

Referring still to FIG. 3, second circuit assembly 170 may be electrically coupled to first circuit assembly 140 along third segment 176, and to a wire assembly 180 along first segment 172, such that second circuit assembly 170 may be configured and operable as an interposer between first circuit assembly 140 and wire assembly 180. Wire assembly 180 may include a plurality of coaxial cables and/or wires that may be electrically coupled to the control system (not shown) of medical device 100 that is operable to control operation of the one or more systems, devices, and/or mechanisms of modular assembly 120. As described above, the control system may include a circuit board disposed within handle 102, which may be in communication with the umbilicus (not shown) connected to medical device 100 via first port 106.

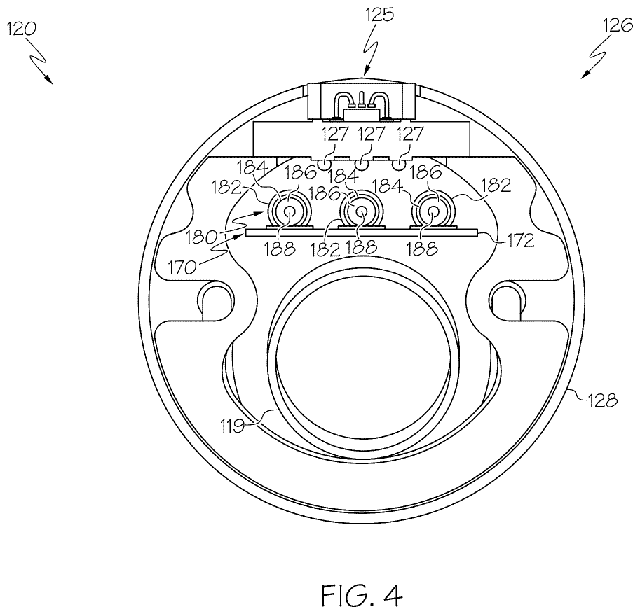

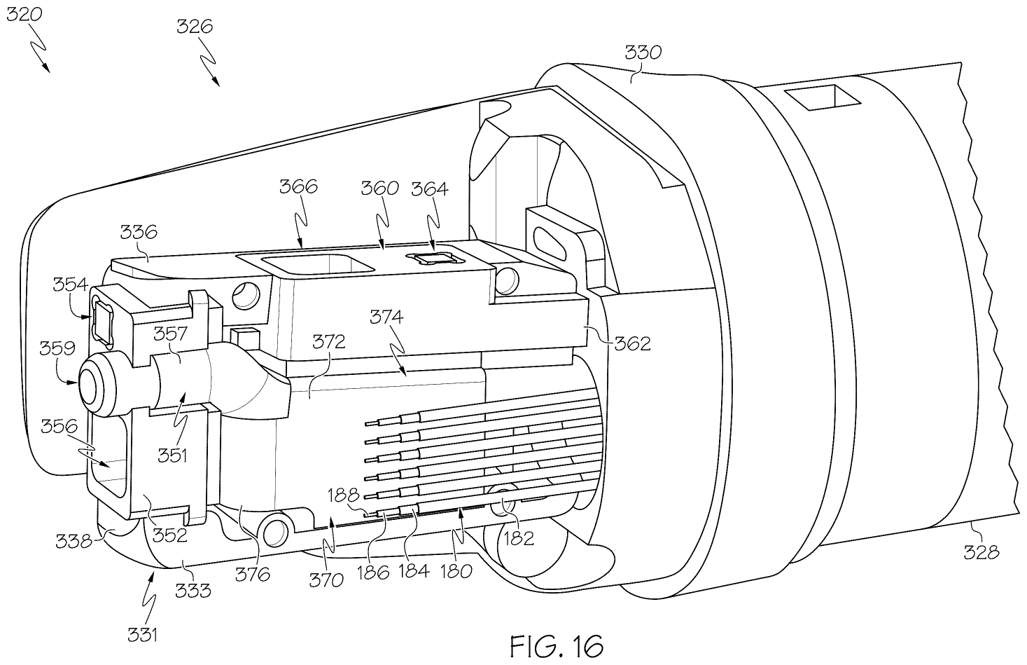

For example, as seen in FIG. 4, wire assembly 180 may include a plurality of coaxial cables, each of which may include an outer insulating layer 182, an outer conducting layer 184, an inner insulating layer 186, and an inner conducting layer 188 that is electrically coupled to first segment 172 of second circuit assembly 170. In some embodiments, outer insulating layer 182 may include a protective cover (e.g., plastic jacket), and outer conducting layer 184 may include a conductor (e.g., metallic shield) that shields inner conducting layer 188 from electrical noise near wire assembly 180, such as to prevent the electrical noise from interfering with an electrical transmission of wire assembly 180. Further, inner insulating layer 186 may include a dielectric insulator that separates outer conducting layer 184 from inner conducting layer 188, and inner conducting layer 188 may include an inner wire and/or conductor that is configured to carry the electrical transmission between second circuit assembly 170 and the control system. In other embodiments, the plurality of coaxial cables may include additional and/or fewer layers than those shown and described herein without departing from a scope of this disclosure. In the example, wire assembly 180 may include three coaxial cables, while in other examples wire assembly 180 may include additional and/or fewer coaxial cables (see FIGS. 16-19).

Referring to FIGS. 5-6, each of first segment 172, second segment 174, and third segment 176 may have varying shapes, sizes, and/or cross-sectional profiles relative to one another to accommodate receipt and/or connection with the respective components of modular assembly 120 described above. First segment 172 may be sized, shaped, and/or otherwise configured to receive wire assembly 180 thereon, such that a cross-sectional profile of first segment 172 may be sufficiently sized to receive the plurality of coaxial cables of wire assembly 180. Third segment 176 may be sized, shaped, and/or otherwise configured to receive first circuit assembly 140 thereon, such that a cross-sectional profile of third segment 176 may be sufficiently sized to receive body 142 of first circuit assembly 140. Second segment 174 may be sized, shaped, and/or otherwise configured to have a cross-sectional profile that is smaller than first segment 172 and third segment 176.

It should be appreciated that first segment 172, second segment 174, and third segment 176 are depicted in FIGS. 5-6 in a flattened configuration prior to assembling modular tip assembly 120 with second circuit assembly 170 in its assembled arrangement, as seen in FIGS. 2-3. As described above, second segment 174 may extend in a transverse direction (non-zero angle) relative to the longitudinal axis of modular assembly 120, such that second segment 174 is oriented in an angled configuration relative to first segment 172. It should be appreciated that second segment 174 may be angled to a degree to accommodate a transverse arrangement of third segment 176 relative to first segment 172. In this instance, second segment 174 may accommodate an orientation of third segment 176 to provide a distal-facing arrangement for modular assembly 120, as best seen in FIGS. 2-3.

Still referring to FIGS. 5-6, second circuit assembly 170 may include one or more printed circuit board (PCB) traces along one or more of first segment 172, second segment 174, and/or third segment 176. The one or more PCB traces may define a network of conductive tracks for electrically coupling second circuit assembly 170 to the one or more components of modular assembly 120, such as wire assembly 180 and first circuit assembly 140. For example, first segment 172 may include a plurality of first PCB traces 171 that are sized, shaped, and/or otherwise configured to receive and/or electrically couple with the plurality of coaxial cables of wire assembly 180. For example, first PCB traces 171 may define elevated pads that are securely attached (e.g., soldered) to wires 188 of wire assembly 180. Second segment 174 may include a plurality of second PCB traces 175 that extend between the plurality of first PCB traces 171 on first segment 172 and a corresponding plurality of third PCB traces on third segment 176 (not shown). The plurality of third PCB traces on third segment 176 may be sized, shaped, and/or otherwise configured to electrically couple with first circuit assembly 140 and the mechanisms mounted thereon. It should be appreciated that additional electronic components and/or mechanisms may be mounted onto second circuit assembly 170, such as along second segment 174, without departing from a scope of this disclosure.

Modular assembly 120 may include one or more material layers disposed inside second channel 136, including outer cover 160, an intermediate layer 162 disposed relatively beneath (proximally to) outer cover 160, and an inner layer 164 disposed relatively beneath (proximally to) intermediate layer 162. In some examples, outer cover 160 may include a light diffuser, intermediate layer 162 may include phosphor, and inner layer 164 may include an optically clear silicone and/or an epoxy. As described above, lighting mechanism 168 may be secured to first circuit assembly 140, such that lighting mechanism 168 may be inserted into end cap 130 when first circuit assembly 140 is assembled onto end cap 130. For example, second channel 136 may be defined by one or more interior walls 166 that terminate at a bottom opening that is sized, shaped, and/or otherwise configured to receive lighting mechanism 168 when end cap 130 is coupled to first circuit assembly 140. In this instance, lighting mechanism 168 may be disposed inside second channel 136, between the one or more interior walls 166, and encapsulated by inner layer 164, intermediate layer 162, and outer cover 160. In other embodiments, inner layer 164 may be omitted entirely such that the phosphor material of intermediate layer 162 may be applied directly on top of lighting mechanism 168.

Still referring to FIGS. 5-6, lighting mechanism 168 may include a light-emitting diode (LED) chip positioned on first circuit assembly 140. For example, lighting mechanism 168 may be wire bonded to first circuit assembly 140 via a bond wire 169. In other embodiments, lighting mechanism 168 may be mounted onto first circuit assembly 140 in a flip chip configuration in which lighting mechanism 168 may include one or more solder pads along a lower surface that interfaces with first circuit assembly 140. In some embodiments, lighting mechanism 168 may include a blue LED chip. In this instance, the blue LED chip of lighting mechanism 168 may be configured and operable to generate a blue light.

In some embodiments, interior walls 166 of second channel 136 may be formed and/or coated with a reflective material, such that second channel 136 may be configured and operable to enhance the light output from lighting mechanism 168 by reflecting the emitted light with interior walls 166. For example, interior walls 166 may be coated, polished, sputtered, or plated with reflective material to provide a high transmissibility of the light within second channel 136. It should be appreciated that end cap 130 may be coupled to first circuit assembly 140 prior to inserting the one or more material layers (e.g., outer cover 160, intermediate layer 162, and inner layer 164) into second channel 136 when assembling modular assembly 120, such that lighting mechanism 168 and bond wire 169 may be encapsulated within second channel 136 by outer cover 160, intermediate layer 162, and inner layer 164.

In the example, the optically clear silicone of inner layer 164 may be configured and operable to form a gap within second channel 136 between lighting mechanism 168 and intermediate layer 162. In other words, the inner layer 164 may be configured to form a remote phosphor configuration in end cap 130 by separating lighting mechanism 168 from intermediate layer 162. The remote phosphor of intermediate layer 162 may configured and operable to modify and/or convert the blue light emitted by the blue LED chip of lighting mechanism 168 to white light prior to exiting second channel 136. The light diffuser of outer cover 160 may configured and operable to evenly diffuse the light emitted out of second channel 136. In other embodiments, outer cover 160 may include a filter, a lens, or various other optical devices.

In the embodiment, by integrating the blue LED chip of lighting mechanism 168 with the optically clear silicone of inner layer 164 and the remote phosphor of intermediate layer 162, modular assembly 120 may be configured and operable to selectively customize the white light that is generated from end cap 130 with adapted optical characteristics that may be personalized for the particular medical procedure. In this instance, different compositions, formulations, and/or amounts of the optically clear silicone of inner layer 164 and the remote phosphor of intermediate layer 162 may be integrated into second channel 136 with the blue LED chip of lighting mechanism 168 to generate the adapted optical characteristics.

For example, a formulation of the remote phosphor of intermediate layer 162 may be selectively customizable to generate an illumination output from end cap 130 with adapted optical characteristics, including but not limited to, a color rendering index (CRI) and a corrected color temperature (CCT). It should be understood that a CRI value may be indicative of a capability for depicting a target object within a procedure site (e.g., an anatomy) in color with detailed accuracy, with a greater CRI value providing a true color representation of the target object. In the example, the formulation of the remote phosphor of intermediate layer 162 may be customized to provide a CRI of modular assembly 120 that may be greater than at least 70, such as greater than about 90. It should further be understood that a CCT value may be indicative of a quality measurement of white light, and more particularly the relative proportions of various colors included in the resulting white light that is emitted from end cap 130. In the example, the formulation of the remote phosphor of intermediate layer 162 may be customized to provide a CCT of modular assembly 120 that may range between about 2200 Kelvin degrees to about 6500 Kelvin degrees.

In other embodiments, the one or more interior walls 166 of second channel 136 may be at least partially formed with phosphor, such that interior walls 166 may be configured and operable to convert the blue light emitted by the blue LED chip of lighting mechanism 168 to white light. Stated differently, phosphor may be mixed with a material composition of interior walls 166, and/or interior walls 166 may be coated with phosphor. In either instance, intermediate layer 162 may be omitted from within second channel 136 when phosphor is integrated into interior walls 166 of second channel 136. Although not shown, it should be understood that end cap 130 may include additional lighting mechanisms 168 (e.g., LEDs) mounted on end cap 130 without departing from a scope of this disclosure. In these embodiments, each lighting mechanism 168 may include a similar or different phosphor formulation to achieve illumination with multiple optical characteristics (e.g., CCT and CRI).

Although the embodiment is described in terms of converting blue light to white light, it should be appreciated that other various colors and/or optical lighting characteristics may be emitted by lighting mechanism 168 and modified by the materials and/or features of end cap 130 without departing from a scope of this disclosure. For example, lighting mechanism 168 may be configured to emit a first light having a first characteristic (e.g., a first color) and intermediate layer 162 and/or interior walls 166 may be operable to modify the first light to a second light having a second characteristic (e.g., a second color) that is different than the first characteristic.

Referring specifically to FIG. 6, and as described above, first circuit assembly 140 may include body 142 that is sized, shaped, and/or otherwise configured to facilitate receipt of one or more mechanisms thereon, including imaging mechanism 150 and lighting mechanism 168. Imaging mechanism 150 may include a camera (e.g., an imager-lens stack) mounted onto first circuit assembly 140, and lighting mechanism 168 may include an LED chip mounted onto first circuit assembly 140. First circuit assembly 140 may be formed of a thermally conductive material (e.g., a substrate), such that first circuit assembly 140 may be configured and operable to manage and/or mitigate a thermal conductivity from imaging mechanism 150 and lighting mechanism 168 during use of modular assembly 120.

For example, the thermally conductive material of first circuit assembly 140 may include, but is not limited to, aluminum nitride (AlN), alumina, silicon nitride, or other suitable materials configured for thermal management. In other examples, end cap 130 and/or sleeve 128 may be formed of a thermally conductive material to facilitate management of thermal heat generated by the one or more mechanisms of modular assembly 120, such as imaging mechanism 150 and lighting mechanism 168, from affecting operation of sensor 125 and to enhance performance of lighting mechanism 168. In further examples, the adhesive inserted into modular assembly 120 for encapsulation of the internal mechanisms and/or assemblies may be thermally conductive and operable to dissipate heat. In additional examples, modular assembly 120 may include a conductive member (e.g., a wire) coupled to first circuit assembly 140 and that is configured to dissipate heat, such as a heat generated from the light emitted by lighting mechanism 168.

Accordingly, one or more of first circuit assembly 140, end cap 130, sleeve 128, and/or the adhesive may be collectively configured to dissipate the heat generated by the mechanisms of modular assembly 120, which may increase an efficiency and/or light output from modular assembly 120. In some embodiments, the material of first circuit assembly 140 (e.g., alumina) may be further configured and operable to enhance a reflectivity of first circuit assembly 140, such as along a portion of first circuit assembly 140 that includes lighting mechanism 168 mounted thereon when coupled within second channel 136 of end cap 130. In further embodiments, end cap 130 and/or sleeve 128 may be radiopaque to facilitate visualization of modular assembly 120 via fluoroscopy. Additionally and/or alternatively, one or more of end cap 130 and/or sleeve 128 may be formed of a metal to inhibit any heat generated therein by the internal mechanisms and/or assemblies from being emitted external to modular assembly 120.

Still referring to FIG. 6, modular assembly 120 may include a coupling capacitor 146 mounted onto the first (distal) face of body 142. Coupling capacitor 146 may be configured and operable to transmit an alternating current (AC) signal from one circuit element to another (e.g., first circuit assembly 140 and second circuit assembly 170) while blocking a direct current (DC) signal from being transmitted. In other words, coupling capacitor 146 may be configured to permit transmission of AC signals while inhibiting transmission of DC signals. It should be understood that AC signals may be indicative of an output signal and DC signals may be indicative of power to the circuit elements to facilitate operation. To preserve values of voltage, current, and/or resistance within first circuit assembly 140 and/or second circuit assembly 170, coupling capacitor 146 may be operable to allow AC variations to be coupled between the circuit assemblies and block any DC coupling therebetween. In other embodiments, end cap 130 may be configured and operable to take out the AC signal and provide a stable power and ground line for modular assembly 120.

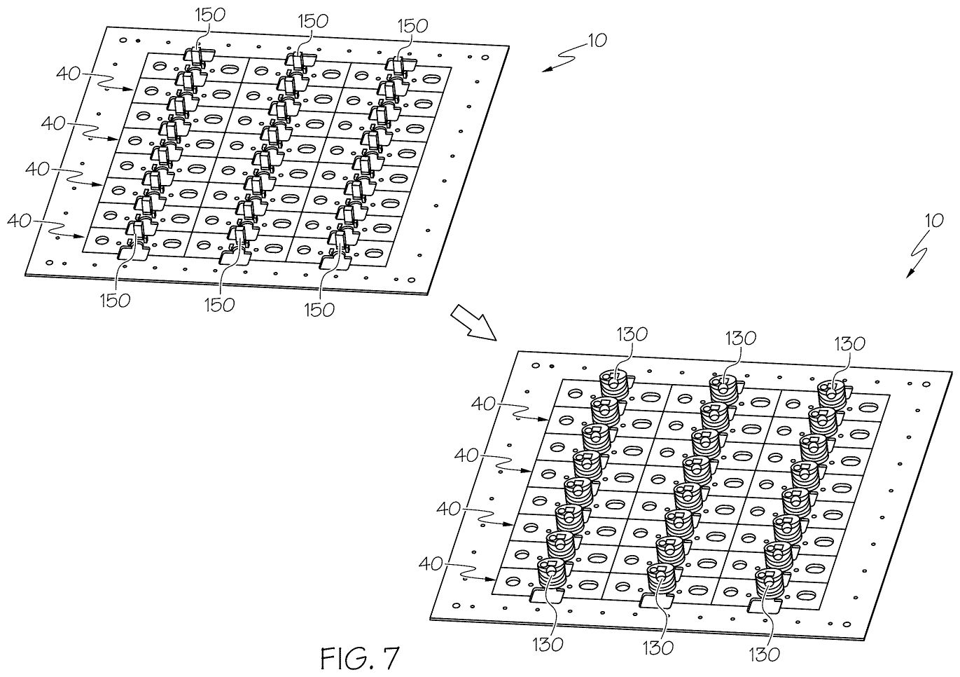

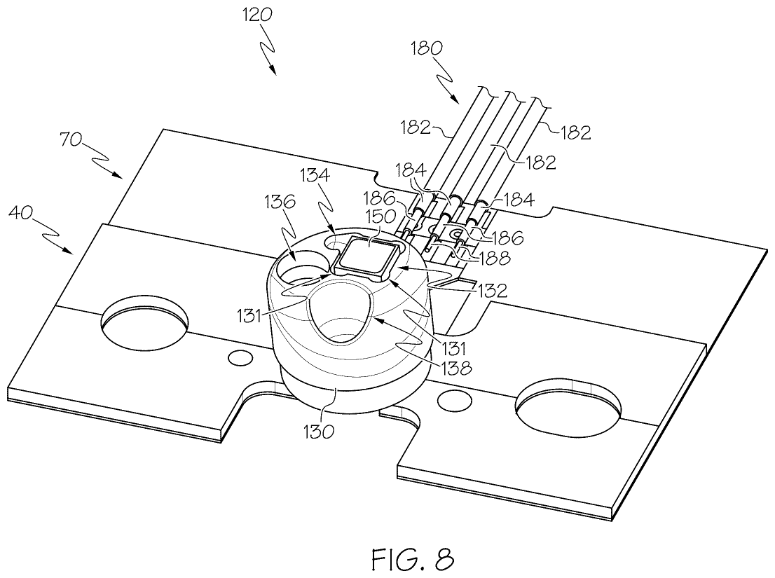

Referring now to FIGS. 7-8, an exemplary method of assembling and/or manufacturing medical device 100, and particularly modular assembly 120, is depicted. In the example, one or more (e.g., a plurality) modular assemblies 120 may be manufactured via an automated assembly in a panelized format. Referring specifically to FIG. 7, a panel 10 may include an array of panelized modules 40 for manufacturing and assembling a plurality of modular assemblies 120 thereon simultaneously. Panel 10 may include a rigid panelized ceramic such that panelized modules 40 may define a corresponding first circuit assembly 140 for each of the plurality of modular assemblies 120 manufactured on the particular panel 10. In this instance, each of the one or more mechanisms and/or components of modular assembly 120 may be assembled onto each panelized module 40, including coupling capacitor 146, imaging mechanism 150, lighting mechanism 168, bond wire 169, etc.

Upon assembling the numerous mechanisms of modular assembly 120 onto the panelized modules 40 of the panel 10, end cap 130 may be securely mounted onto the panelized modules 40 and over the aforementioned mechanisms. As described above, end cap 130 may be secured to the internal mechanisms and first circuit assembly 140 via an adhesive inserted therein via fill port 134, thereby securely coupling end cap 130 to the corresponding panelized module 40. With each of the plurality of end caps 130 securely coupled to the corresponding panelized module 40, individual panelized modules 40 may be selectively detached from panel 10. In some embodiments, the panelized modules 40 may be preformed within the panel 10 and easily detachable along one or more borders and/or edges (“tear lines”). In this instance, the panelized modules 40, with end cap 130 and the internal mechanisms coupled thereto, may be decoupled from the panel 10.

Referring now to FIG. 8, each individual panelized module 40 may be securely coupled to a respective flexible panel 70, with a corresponding wire assembly 180 coupled to flexible panel 70. It should be appreciated that wire assembly 180 may be passed through articulation segment 122 prior to integrating articulation segment 122 with shaft 116 (FIG. 1). In this instance, an assembly of modular assembly 120 may be further simplified by not requiring that the plurality of coaxial cables of wire assembly 180 be strung to modular assembly 120 after articulation segment 122 is integrated into distal portion 118 of shaft 116. Stated differently, the method may include assembling modular assembly 120, and particularly end cap 130, sleeve 128, articulation segment 122, and the numerous mechanisms and assemblies disposed therein, into a subassembly prior to integrating the subassembly with shaft 116. Articulation segment 122 may be coupled to one or more steering wires extending through shaft 116, and selectively controlled by one or more actuators 104 on handle 102.

Upon coupling panelized module 40 to flexible panel 70, panelized module 40 may be excised to form first circuit assembly 140 and flexible panel 70 may be excised to form second circuit assembly 170, and particularly the respective sizes, shapes, and/or configurations of each circuit assembly. Stated differently, a size, a shape, and/or a configuration of panelized module 40 may be selectively modified to form body 142 of first circuit assembly 140 (see FIG. 6), and a size, a shape, and/or a configuration of flexible panel 70 may be selectively modified to form the plurality of segments 172, 174, 176 of second circuit assembly 170.

In this instance, panelized module 40 may be reconfigured to define body 142 of first circuit assembly 140, and flexible panel 70 may be reconfigured to define the plurality of segments 172, 174, 176 of second circuit assembly 170. In some embodiments, panelized module 40 and/or flexible panel 70 may include one or more scored lines for excising the respective circuit assembly. Additionally, a size, a shape, and/or a configuration of second circuit assembly 170 may be selectively modified to form the respective cross-sectional profiles and arrangements of first segment 172, second segment 174, and third segment 176 prior to disposing end cap 130, first circuit assembly 140, and second circuit assembly 170 (collectively tip segment 126) within sleeve 128 (see FIGS. 2-3). In this instance, second circuit assembly 170 may be selectively adjusted to bend, position, and/or orient the segments relative to one another for insertion into sleeve 128.

In other embodiments, panel 10 may include a flexible panelized ceramic such that panelized modules 40 may define a corresponding second circuit assembly 170 for each of the plurality of modular assemblies 120 that are manufactured on the particular panel 10. In this instance, the internal mechanisms, first circuit assembly 140, and end cap 130 of modular assembly 120 may be assembled directly onto second circuit assembly 170.

It should be appreciated that the panelized format for automating the manufacture of modular assembly 120 may allow for multiple layers of mechanisms to be assembled onto the panelized modules 40 of panel 10 simultaneously, and in some embodiments via a single step, thereby enabling a high yielding manufacturing method for modular assembly 120 that is fixture-assisted via the panel 10. Accordingly, the fully automated manufacturing method may provide a highly scalable assembly of modular assembly 120 that may be modular and selectively adjusted to accommodate customized design variations for the placement of internal mechanisms and/or end cap 130. In some embodiments, the panelized format for assembling modular assembly 120 may enable utilization of a mixed-model assembly line in which modular assembly 120 may be manufactured alongside other devices (e.g., medical scopes) assembled concurrently via the panelized format.

In other embodiments, modular assembly 120 may be manufactured to include additional assemblies and/or mechanisms than those shown and described herein without departing from a scope of this disclosure. For example, modular assembly 120 may include multiple first circuit assemblies 140 (e.g., rigid circuit boards) including multiple imaging mechanisms 150 and/or lighting mechanisms 168, and the multiple first circuit assemblies 140 may be mounted onto a single second circuit assembly 170 and/or multiple second circuit assemblies 170 (e.g., flexible circuit boards) for attachment and use with medical device 100.

Referring now to FIGS. 9-13, another exemplary modular assembly 220 is depicted. Modular assembly 220 may be substantially similar to modular assembly 120 except for the differences explicitly described herein. Accordingly, the same reference numerals are used to identify substantially similar components. Additionally, modular assembly 220 may be configured and operable similar to modular assembly 120, such that modular assembly 220 may be integrated onto distal portion 118 of medical device 100 in a substantially similar manner as modular assembly 120 shown and described above.

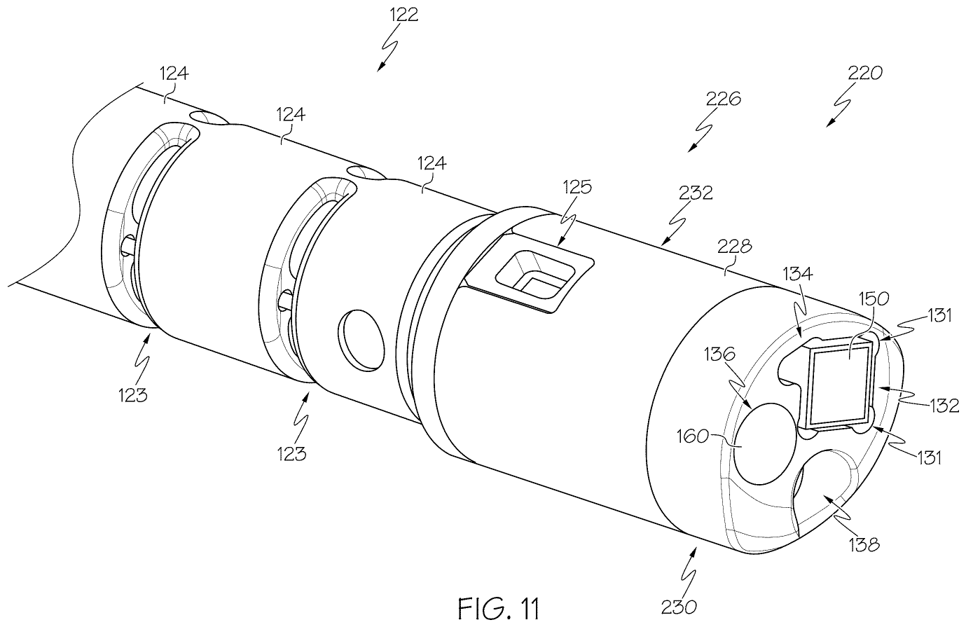

Referring specifically to FIG. 9, modular assembly 220 may include articulation segment 122 with the plurality of links 124 and cutouts 123, and a tip segment 226 with a sleeve 228 and an end cap 230. Sleeve 228 may include an aspiration port 232 formed along an exterior surface of sleeve 228, and modular assembly 220 may include an aspiration channel 234 disposed inside sleeve 228 and in fluid communication with aspiration port 232. It should be appreciated that a distal end of aspiration channel 234 may terminate at aspiration port 232, and aspiration channel 234 may extend from tip segment 226 into articulation segment 122 and through shaft 116 with a proximal end of aspiration channel 234 terminating at and/or adjacent to handle 102 (see FIG. 1). In the example, aspiration port 232 may be formed along a surface of sleeve 228 that is different than a surface of sleeve 228 including sensor 125 (see FIG. 11).

As best seen in FIG. 10, modular assembly 220 may include a fill port 231 formed along an exterior surface of sleeve 228. Fill port 231 may be sized, shaped, and/or otherwise configured to receive a material (e.g., an adhesive) into sleeve 228 to securely couple the internal mechanisms and/or assemblies to one another. Additionally and/or alternatively, the material received via fill port 231 may be operable to waterproof the interior cavities of tip segment 226. In other embodiments, modular assembly 220 may include additional and/or fewer fill ports 231 positioned along various other portions of sleeve 228 and/or end cap 230.

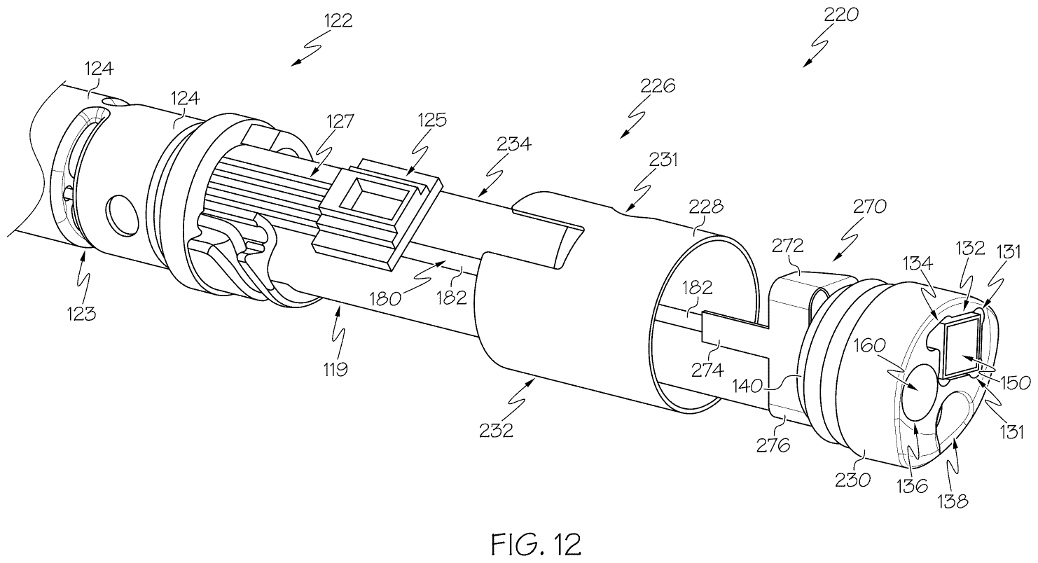

Referring now to FIG. 12, modular assembly 220 may include first circuit assembly 140 coupled to end cap 230, and a second circuit assembly 270 coupled to a side of first circuit assembly 140 opposite from end cap 230. Second circuit assembly 270 may be substantially similar to second circuit assembly 170 such that the same reference numerals are used to identify substantially similar components. Additionally, second circuit assembly 270 may be configured and operable similar to second circuit assembly 170. In the example, second circuit assembly 270 may be coupled to wire assembly 180. Second circuit assembly 270 may include a first segment 272 and a second segment 274, each of which may be coupled to a third segment 276. Third segment 276 may be further coupled to first circuit assembly 140. In the example, third segment 276 may be selectively positioned in a C-shaped and/or U-shaped configuration, with first segment 272 bent and/or folded relative to third segment 276 in a transverse arrangement (e.g., a 90-degree angle).

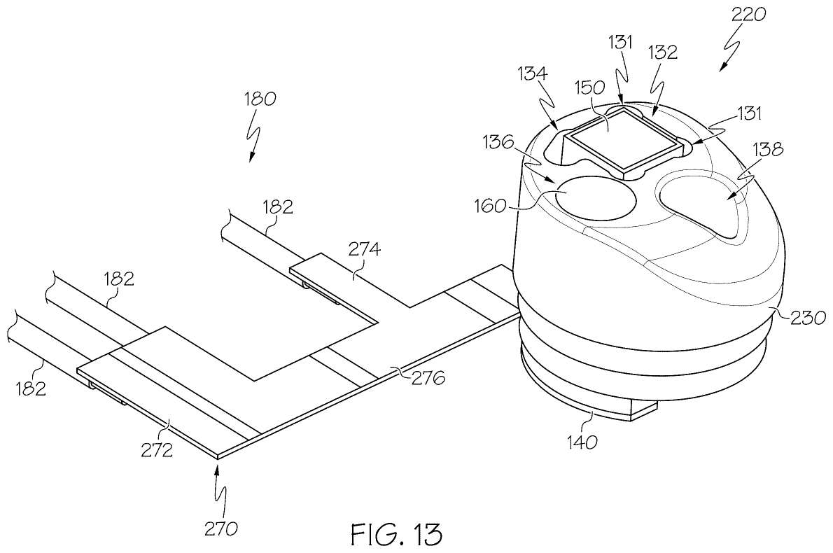

As best seen in FIG. 13, second circuit assembly 270 may include a first segment 272 and a second segment 274, each of which may be coupled to a third segment 276. Third segment 276 may be further coupled to first circuit assembly 140. It should be appreciated that first segment 272, second segment 274, and third segment 276 are depicted in FIG. 13 in a flattened configuration prior to assembling modular tip assembly 220 with second circuit assembly 270 in its assembled arrangement, as seen in FIG. 12. In the example, first segment 272 may be sized, shaped, and/or otherwise configured to couple with a pair of coaxial cables 182 of wire assembly 180, and second segment 274 may be sized, shaped, and/or otherwise configured to couple with a single coaxial cable 182 of wire assembly 180. Accordingly, first segment 272 may have a greater width than second segment 274. It should be appreciated that second circuit assembly 270 may include an arrangement of the segments to accommodate a customized arrangement of wire assembly 180 and/or first circuit assembly 140 within sleeve 228 and/or relative to one another.

Referring now to FIGS. 14-19, another exemplary modular assembly 320 is depicted. Modular assembly 320 may be substantially similar to modular assembly 120 except for the differences explicitly described herein. Accordingly, the same reference numerals are used to identify substantially similar components. Additionally, modular assembly 320 may be configured and operable similar to modular assembly 120, such that modular assembly 320 may be integrated onto distal portion 118 of medical device 100 in a substantially similar manner as modular assembly 120 shown and described above.

Referring specifically to FIG. 14, modular assembly 320 may include articulation segment 122 with the plurality of links 124 and cutouts 123, and a tip segment 326 with a sleeve 328 and an end cap 330. Sleeve 328 may be coupled to end cap 330 for housing one or more mechanisms and/or assemblies therein, and sleeve 328 may be configured in a substantially similar manner as sleeve 128 shown and described above. End cap 330 may include a first (side-facing) opening 332 facing a first (lateral/radially outward) direction and a second (forward-facing) opening 334 facing a second (distal) direction that is different than the first direction. Modular assembly 320 may further include an elevator 340 movably disposed within end cap 330, and elevator 340 may be configured and operable to receive medical instrument 110 thereon (see FIG. 1).

It should be appreciated that elevator 340 may be aligned and in communication with a working channel within shaft 116 of medical device 100, such that such that medical instrument 110 received through the working channel may extend outwardly from end cap 330 along elevator 340. In a first (undeflected) position, elevator 340 may be configured to guide medical instrument 110 out of end cap 330 in the second (distal) direction via second opening 334. In a second (deflected) position, elevator 340 may be configured to guide medical instrument 110 out of end cap 330 in the first (lateral) direction via first opening 332.

End cap 330 may include a first (lateral) surface 336 that is positioned adjacent to first (lateral) opening 332 and facing the first (lateral) direction, and a second (distal) surface 338 that positioned adjacent to second (distal) opening 334 and facing the second (distal) direction. Each of surfaces 336, 338 may include openings for receiving a circuit assembly (e.g., rigid circuit board). For example, second surface 338 may include a first circuit assembly 350 and first surface 336 may include a second circuit assembly 360, each of which may include a rigid circuit board. First circuit assembly 350 may include an imaging mechanism 354 (e.g., a camera), a channel 356, and an irrigation nozzle 359 facing the second (distal) direction. Second circuit assembly 360 may include an imaging mechanism 364 (e.g., a camera) and a channel 366 facing the first (lateral) direction.

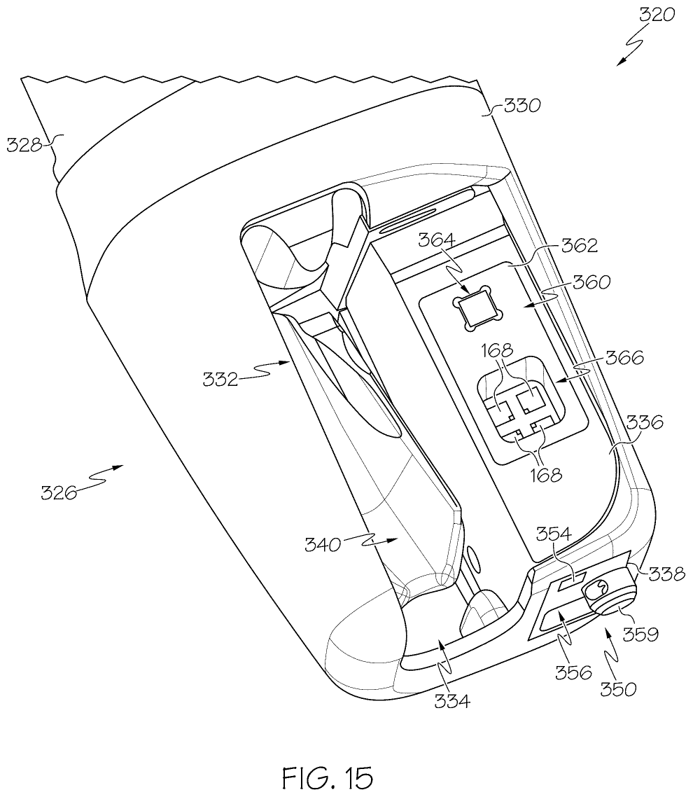

Each of imaging mechanisms 354, 364 may be configured and operable similar to imaging mechanism 150 shown and described in detail above. Each of channels 356, 366 may include one or more lighting mechanisms disposed therein. For example, as best seen in FIG. 15, channel 366 may include a plurality of lighting mechanisms 168, such as four lighting mechanisms 168. In some examples, channel 356 may include a corresponding number of lighting mechanisms 168 as disposed in channel 366.

Modular assembly 320 may include an irrigation channel 357 that is fluidly coupled to irrigation nozzle 359. Irrigation nozzle 359 may be fluidly coupled to irrigation channel 357, and configured and operable to irrigate one or more regions and/or components of modular assembly 320 with a fluid received from irrigation channel 357. For example, irrigation nozzle 359 may include an opening that is angled towards imaging mechanism 354 such that irrigation nozzle 359 may be configured to irrigate a front lens of imaging mechanism 354. As described in further detail herein, irrigation channel 357 may extend through first circuit assembly 350 with a distalmost end of irrigation nozzle 359 positioned external to and distal to first circuit assembly 350 and second (distal) surface 338.

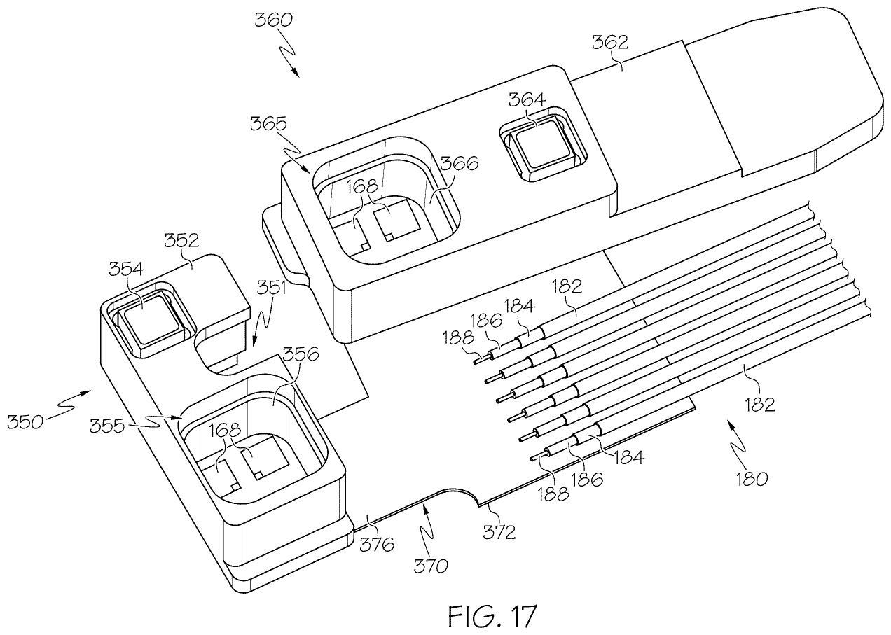

Still referring to FIG. 16, first circuit assembly 350 may include a body 352 housing imaging mechanism 354 and the plurality of lighting mechanisms 168 within channel 356 (see FIG. 17). Modular assembly 320 may include one or more material layers disposed inside channel 356, including an outer cover 355 (see FIG. 17) that is substantially similar to outer cover 160, an intermediate layer (not shown) disposed relatively beneath outer cover 355 that is substantially similar to intermediate layer 162, and an inner layer (not shown) disposed relatively beneath the intermediate layer that is substantially similar to inner layer 164. Body 352 may define a gap 351 that may be configured to receive irrigation channel 357 and irrigation nozzle 359 through first circuit assembly 350.

Second circuit assembly 360 may include a body 362 housing imaging mechanism 364 and the plurality of lighting mechanisms 168 within channel 366. Modular assembly 320 may include one or more material layers disposed inside channel 366, including an outer cover 365 (see FIG. 17) that is substantially similar to outer cover 160, an intermediate layer (not shown) disposed relatively beneath outer cover 365 that is substantially similar to intermediate layer 162, and an inner layer (not shown) disposed relatively beneath the intermediate layer that is substantially similar to inner layer 164.

Still referring to FIG. 16, modular assembly 320 may include a third circuit assembly 370 (e.g., a flexible circuit board) that is coupled to each of first circuit assembly 350 and second circuit assembly 360 along different segments of third circuit assembly 370. In the example, third circuit assembly 370 may include a plurality of segments, including but not limited to, a first (proximal) segment 372, a second (lateral) segment 374 that is coupled and positioned perpendicularly/angled relative to first segment 372, and a third (distal) segment 376 that is coupled and positioned distally relative to first segment 372. In some embodiments, each of first segment 372, second segment 374, and third segment 376 may be integral with one another, such that the plurality of segments of third circuit assembly 370 may have a unitary body. In other embodiments, one or more of first segment 372, second segment 374, and/or third segment 376 may be coupled and/or attached to one another. Further details of third circuit assembly 370 are described below.

In the example, the plurality of coaxial cables of wire assembly 180 may be coupled to third circuit assembly 370 along first segment 372, first circuit assembly 350 may be coupled to third circuit assembly 370 at third segment 376, and second circuit assembly 360 may be coupled to third circuit assembly 370 at second segment 374. In this instance, third circuit assembly 370 may be configured and operable as an interposer between first circuit assembly 350, second circuit assembly 360, and wire assembly 180. First segment 372 may have a longitudinal length that extends in a substantially parallel direction relative to a longitudinal axis of modular assembly 320. In this instance, upper and lower surfaces of first segment 372 may be oriented to face opposing lateral directions within end cap 330.

Still referring to FIG. 16, second segment 374 may have a longitudinal length that extends in a substantially parallel direction relative to the longitudinal axis of modular assembly 320, with upper and lower surfaces of second segment 374 oriented to face opposing directions that are transverse to the longitudinal axis of modular assembly 320. Third segment 376 may have a longitudinal length that extends in a substantially transverse direction relative to the longitudinal axis of modular assembly 320, such that front and rear surfaces of third segment 376 may be oriented to face opposing directions that are parallel to the longitudinal axis of modular assembly 320. Stated differently, a normal line of second segment 374 may extend in a first direction such that a face of second segment 374 extends in the first direction. A normal line of third segment 376 may extend in a second direction that is different than the first direction such that a face of third segment 376 may face the second direction.

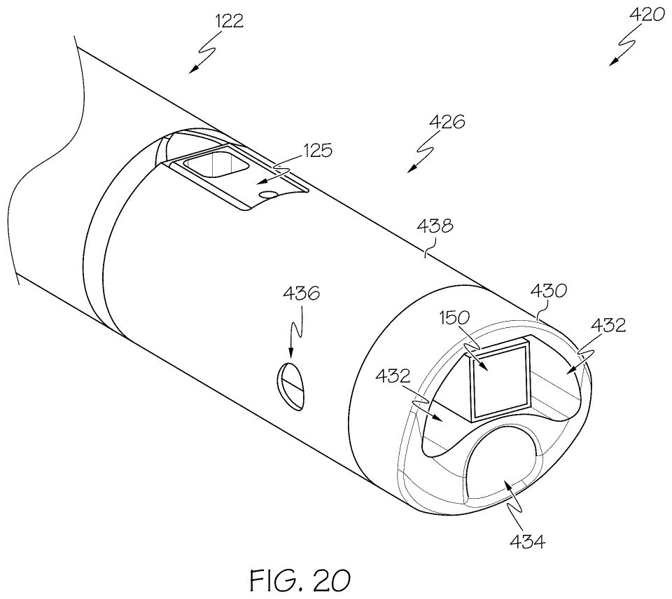

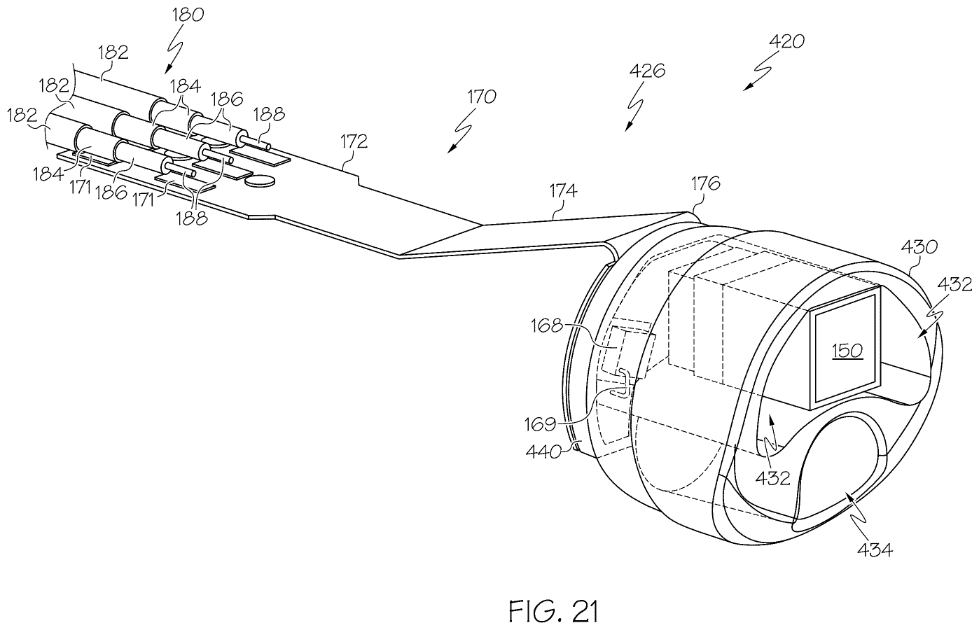

It should be appreciated that a relative configuration of third circuit assembly 370, and particularly the arrangement of first segment 372, second segment 374, and/or third segment 376 relative to one another, may be selectively adjusted to various other suitable positions based on a modular configuration of modular assembly 320 (see FIGS. 20-21). Additionally, third circuit assembly 370 may include additional and/or fewer segments than those shown and described herein without departing from a scope of this disclosure.

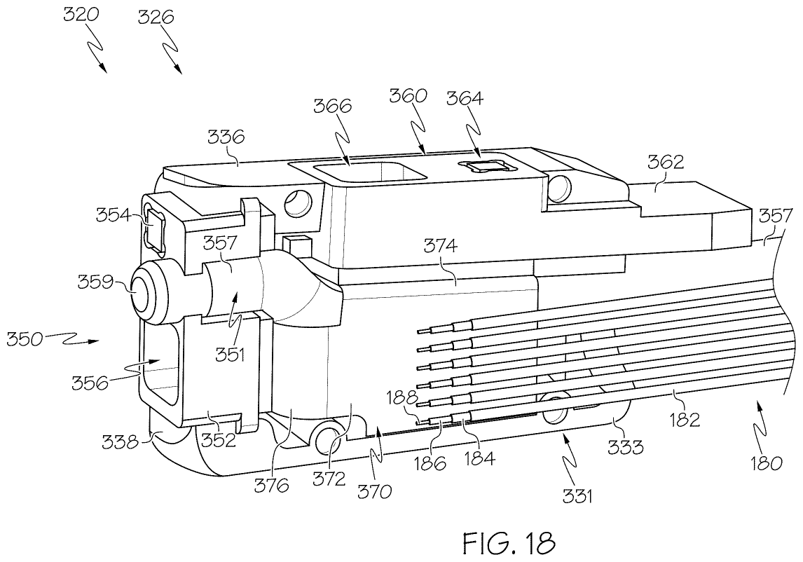

It should be appreciated that modular assembly 320 may be assembled and manufactured in a substantially similar manner as the panelized format shown and described above with respect to modular assembly 120 (see FIGS. 7-8) except for the differences explicitly noted herein. For example, referring now to FIG. 17, each of wire assembly 180, first circuit assembly 350, and second circuit assembly 360 may be assembled onto a panelized module defining third circuit assembly 370. In this instance, each modular assembly 320 may be assembled onto a flexible panelized board such that the panelized modules define a corresponding third circuit assembly 370 for each of the plurality of modular assemblies 320 manufactured on a particular panel 10. It should be appreciated that first segment 372, second segment 374, and third segment 376 are depicted in FIG. 17 in a flattened configuration prior to assembling modular tip assembly 320 with circuit assemblies 350, 360 in its assembled arrangement, as seen in FIG. 18.

In addition to assembling wire assembly 180, first circuit assembly 350, and second circuit assembly 360 onto third circuit assembly 370, the multiple components and/or mechanisms of each assembly may be assembled onto the respective assembly. For example, imaging mechanism 354 may be assembled onto body 352 and lighting mechanisms 168 may be assembled into channel 356 of first circuit assembly 350. Imaging mechanism 364 may be assembled onto body 362 and lighting mechanisms 168 may be assembled into channel 366 of second circuit assembly 360. The one or more material layers may be integrated into the respective channels 356, 366 of the assemblies 350, 360, as described in detail above.

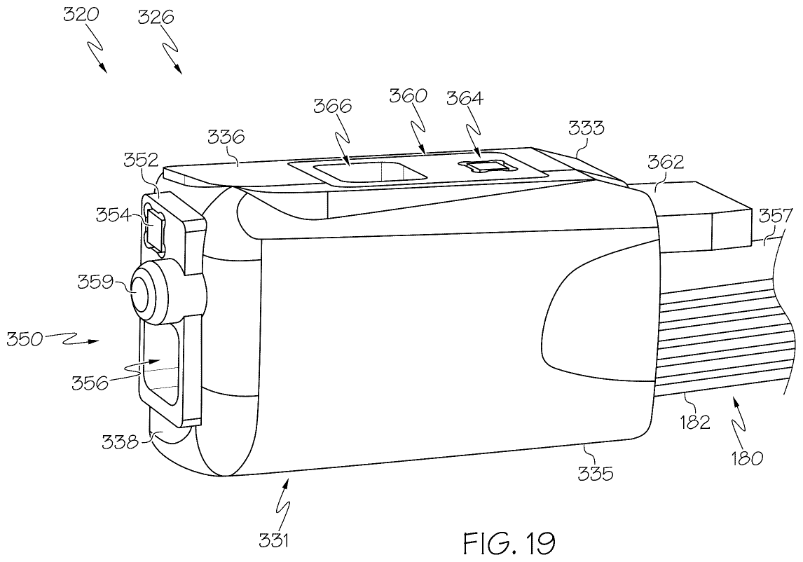

Referring now to FIGS. 18-19, wire assembly 180, circuit assemblies 350, 360, 370, and irrigation channel 317 may be integrated into a housing 331. Housing 331 may include a first shell 333 and a second shell 335 (see FIG. 19) that may be collectively sized, shaped, and/or otherwise configured to house wire assembly 180, circuit assemblies 350, 360, 370, and irrigation channel 317 to one another in the customized arrangement. First shell 333 and second shell 335 may be configured to attach to one another with wire assembly 180, circuit assemblies 350, 360, 370, and irrigation channel 317 securely disposed therein. Upon assembly, housing 331 may be filled with a material (e.g. adhesive) to secure first shell 333 to second shell 335, and encapsulate the internal mechanisms and/or assemblies therein. Housing 331 may be received within and/or coupled to end cap 330, and end cap 330 may be further coupled to sleeve 328, thereby completing assembly of tip segment 326 of modular assembly 320 for attachment to articulation segment 122.

Referring now to FIGS. 20-22, another exemplary modular assembly 420 is depicted. Modular assembly 420 may be substantially similar to modular assembly 120 except for the differences explicitly described herein. Accordingly, the same reference numerals are used to identify substantially similar components. Additionally, modular assembly 420 may be configured and operable similar to modular assembly 120, such that modular assembly 420 may be integrated onto distal portion 118 of medical device 100 in a substantially similar manner as modular assembly 120 shown and described above.

Referring specifically to FIG. 20, modular assembly 420 may include articulation segment 122 and a tip segment 426. In some embodiments, articulation segment 122 may include a tungsten braid to facilitate visualization of articulation segment 122 in fluoroscopy. In the example, articulation segment 122 may be coupled (e.g., glued, thermally bonded, etc.) to tip segment 426. Although not shown, it should be understood that articulation segment 122 of modular assembly 420 may include the plurality of links 124 and corresponding cutouts 123 as shown and described in detail above. Alternatively, it should be understood that the articulation of the other modular assemblies described above and further herein may include the tungsten braid without departing from a scope of this disclosure.

Tip segment 426 may include a sleeve 438 and an end cap 430. Sleeve 428 may be coupled to end cap 430 for housing one or more mechanisms and/or assemblies therein, and sleeve 438 may be configured in a substantially similar manner as sleeve 128 shown and described above. In the example, sleeve 438 may include a fill port 436 along an exterior surface of sleeve 438 for receiving a material (e.g. adhesive) to encapsulate internal mechanisms and waterproof sleeve 438. In some embodiments, sleeve 438 may be formed of a stainless steel material to serve as a heat sink, thereby minimizing heating effects on sensor 125 and enhancing a lighting performance of modular assembly 420. In further embodiments, sleeve 438 may be coated and/or formed of a radiopaque material to facilitate visualization of modular assembly 420 via fluoroscopy.

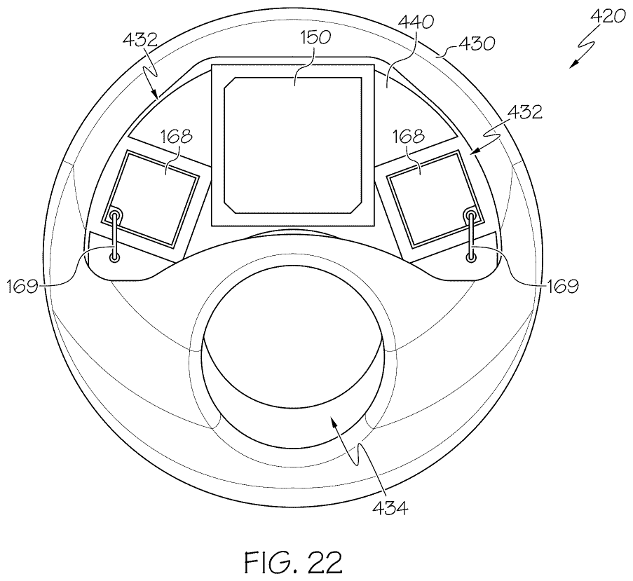

Still referring to FIG. 20, end cap 330 may include a first channel 432 and a second channel 434 disposed relatively beneath first channel 432. First channel 432 may be sized, shaped, and/or otherwise configured to receive imaging mechanism 150 and one or more lighting mechanisms 168 therein, as best seen in FIG. 22. Second channel 434 may be aligned with the working channel of shaft 116 when modular assembly 420 is coupled to distal portion 118 (see FIG. 1). Modular assembly 420 may be configured to receive one or more layers of material within first channel 432 to encapsulate imaging mechanism 150 and the one or more lighting mechanisms 168 (FIG. 22) therein. For example, the one or more material layers may include a material that is configured and operable to diffuse light emitted by lighting mechanisms 168, a remote phosphor material, and an optically clear silicone and/or an epoxy, as described above.

In some embodiments, end cap 430 may be formed of a clear polycarbonate material such that modular assembly 420 may be configured to transmit light from lighting mechanisms 168 and out of end cap 430 towards various relative directions (e.g., distally, laterally, etc.). Additionally, the clear polycarbonate material of end cap 430 may be operable to inhibit heat generation from the light emitted by lighting mechanisms 168, thereby maintaining a temperature of end cap 430.

Referring now to FIG. 21, modular assembly 420 may include a first circuit assembly 440 coupled to end cap 430 with imaging mechanism 150 and the one or more lighting mechanisms 168 disposed within the same channel 432. Second circuit assembly 170 may be coupled to first circuit assembly 440 along third segment 176, and wire assembly 180 may be coupled to second circuit assembly 170 along first segment 172. In the example, as best seen in FIG. 22, modular assembly 420 may include a pair of lighting mechanisms 168 coupled to first circuit assembly 440 and disposed within first channel 432 along opposing sides of imaging mechanism 150. In some embodiments, with end cap 430 formed of the clear polycarbonate material, first circuit assembly 440 may be configured and operable as a reflector. For example, first circuit assembly 440 may be formed of alumina, or may be coated with a metallization material (e.g., gold, copper, etc.), to enhance a reflectivity of first circuit assembly 440 through end cap 430.

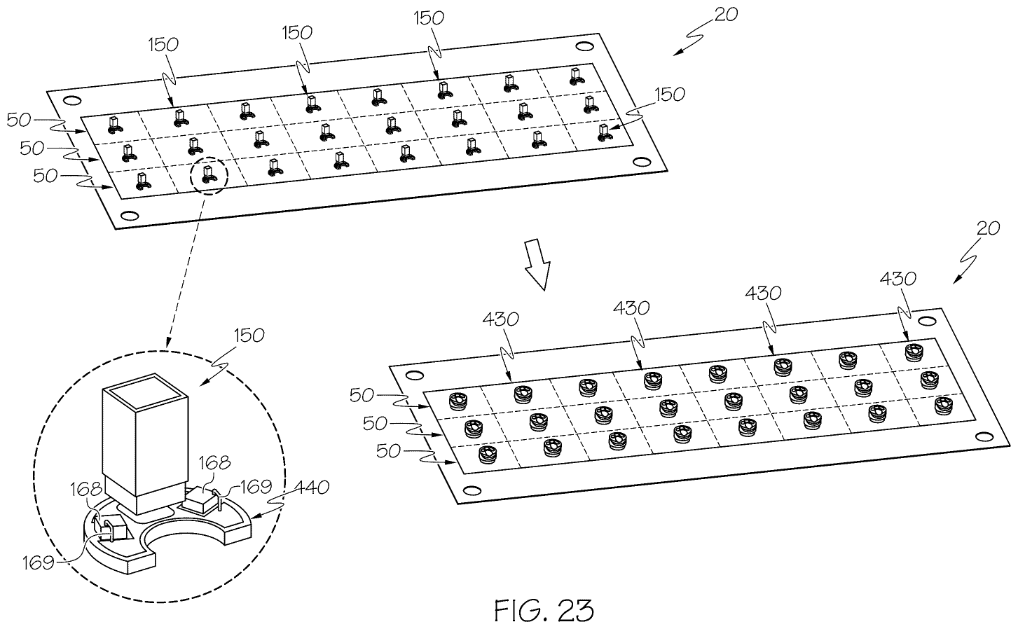

It should be appreciated that modular assembly 420 may be assembled and manufactured in a substantially similar manner as the panelized format shown and described above with respect to modular assembly 120 (see FIGS. 7-8) except for the differences explicitly noted herein. For example, referring now to FIG. 23, each of first circuit assembly 440, imaging mechanism 150, and lighting mechanisms 168 may be assembled onto a panelized module 50 of a panel 20 that defines second circuit assembly 170. In this instance, each modular assembly 420 may be assembled onto a flexible panelized board such that the panelized modules 50 of the panel 20 define a corresponding second circuit assembly 170 for each of the plurality of modular assemblies 420 manufactured on the particular panel 20.

In some embodiments, imaging mechanism 150 and lighting mechanisms 168 may be initially assembled onto first circuit assembly 440, and first circuit assembly 440 may be coupled onto the panelized module 50 on the panel 20. In this instance, end cap 430 may be coupled over first circuit assembly 440 with imaging mechanism 150 and lighting mechanisms 168 secured thereto, and a material may be received within end cap 430 to encapsulate the mechanisms therein. The panelized module 50 may be detached from the panel 20 and excised to form the customized configuration of second circuit assembly 170, and particularly the plurality of segments of second circuit assembly 170. It should be appreciated that the plurality of panelized modules 50 on the panel 20 may each define a corresponding second circuit assembly 170 for a plurality of modular assemblies 420.

Each of the aforementioned systems, devices, assemblies, and methods may be used to treat a target treatment site with a medical device including a modular tip assembly. By providing a medical device with a modular tip assembly, treatment of a subject (e.g., patient) may be achieved with a high degree of customization and design flexibility through use of the modular tip assembly that is configured for use in the procedure.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a handle;
a shaft extending distally from the handle; and
an assembly coupled to a distal end of the shaft, wherein the assembly includes:
an end cap including a first channel and a second channel;
an imaging mechanism coupled to the end cap and received within the first channel;
a lighting mechanism coupled to the end cap and received within the second channel;
a first circuit assembly coupled to the end cap, wherein the imaging mechanism and the lighting mechanism are mounted onto the first circuit assembly; and
a second circuit assembly coupled to the first circuit assembly, the second circuit assembly is flexibly deformable to selectively position the first circuit assembly in alignment with the end cap;
wherein the end cap includes a material received within the second channel and disposed over the lighting mechanism, the material being configured to modify a light emitted outwards from the end cap by the lighting mechanism; and
wherein the first circuit assembly includes a rigid circuit board communicatively coupled to the imaging mechanism and the lighting mechanism, and the second circuit assembly includes a flexible circuit board interposed between and communicatively coupled to the first circuit assembly and a control system of the medical device.

2. The medical device of claim 1, wherein the end cap includes a port positioned adjacent to and in fluid communication with the first channel, wherein the port is configured to receive a second material that extends into the first channel to securely attach the imaging mechanism to the first channel.

3. The medical device of claim 2, wherein the end cap includes a plurality of divots positioned about and in fluid communication with the first channel, the plurality of divots defining a gap that extends about a perimeter of the imaging mechanism within the first channel.

4. The medical device of claim 3, wherein the gap is configured to receive the second material from the port, thereby encapsulating the imaging mechanism with the second material.

5. The medical device of claim 1, wherein the assembly includes a sensor configured to detect a pressure applied to the distal end of the shaft.

6. The medical device of claim 1, wherein the end cap is formed of a thermally conductive material that is configured to dissipate heat generated by the lighting mechanism and the imaging mechanism.

7. The medical device of claim 1, wherein the flexible circuit board includes a first segment coupled to a wire assembly that is communicatively coupled to the control system, a second segment coupled to the rigid circuit board, and a third segment positioned between the first segment and the second segment.

8. The medical device of claim 7, wherein the first segment, the second segment, and the third segment are individually movable relative to one another to arrange the wire assembly and the rigid circuit board at predefined configurations relative to one another.

9. The medical device of claim 1, wherein the material includes a first material, and the end cap includes a second material received within the second channel and disposed between the first material and the lighting mechanism, such that the second material is configured to separate the first material from the lighting mechanism.

10. The medical device of claim 9, wherein the end cap includes a third material received within the second channel and disposed over the first material, the third material is configured to evenly diffuse the light emitted by the lighting mechanism and modified by the first material when exiting the first channel.

11. The medical device of claim 10, wherein the first material includes a phosphor layer, the second material includes a silicone layer, and the third material includes a diffuser layer.

12. The medical device of claim 1, wherein the lighting mechanism includes a light-emitting diode that is configured to emit blue light, and the material includes a phosphor formulation that is configured to modify the blue light to white light.

13. The medical device of claim 1, wherein the shaft includes an articulation segment at the distal end of the shaft that is configured to move the assembly relative to the shaft.

14. The medical device of claim 13, wherein the assembly includes a sleeve coupled between the end cap and the articulation segment, the sleeve is configured to house the first circuit assembly and the second circuit assembly;
wherein the sleeve includes an aspiration port that is in fluid communication with an aspiration channel of the shaft.

15. A medical device, comprising:
a handle;
a shaft extending distally from the handle; and
an assembly coupled to a distal end of the shaft, wherein the assembly includes:
an end cap;
a pair of first circuit assemblies coupled to the end cap, wherein each of the pair of first circuit assemblies includes an imaging mechanism and a lighting mechanism mounted onto the first circuit assembly; and
a second circuit assembly coupled to each of the pair of first circuit assemblies, wherein the second circuit assembly is flexibly deformable to selectively position the pair of first circuit assemblies in predefined configurations relative to the end cap;
wherein each of the pair of first circuit assemblies includes a material disposed over the lighting mechanism, the material being configured to modify a light emitted outwards from the end cap by each lighting mechanism from the pair of first circuit assemblies; and
wherein the pair of first circuit assemblies include rigid circuit boards and the second circuit assembly includes a flexible circuit board.

16. The medical device of claim 15, wherein each of the pair of first circuit assemblies includes a channel configured to receive the imaging mechanism and a port in fluid communication with the channel, the port is configured to guide a second material into the channel to securely attach the imaging mechanism to the channel;
wherein each of the pair of first circuit assemblies includes a plurality of divots in fluid communication with the channel and defining a gap about a perimeter of the imaging mechanism within the channel, the gap is configured to receive the second material from the port, thereby encapsulating the imaging mechanism with the second material.

17. The medical device of claim 15, wherein each of the pair of first circuit assemblies includes a rigid circuit board communicatively coupled to the imaging mechanism and the lighting mechanism, and the second circuit assembly includes a flexible circuit board interposed between and communicatively coupled to the pair of first circuit assemblies and a control system of the medical device.

18. The medical device of claim 15, wherein the material includes a first material, and the end cap includes a second material disposed between the first material and the lighting mechanism to separate the first material from the lighting mechanism, and a third material disposed over the first material that is configured to evenly diffuse the light emitted by the lighting mechanism and modified by the first material upon exiting the end cap.

19. The medical device of claim 18, wherein the first material includes a phosphor layer, the second material includes a silicone layer, and the third material includes a diffuser layer.

20. A method of assembling a plurality of medical devices, comprising:
coupling a first circuit assembly onto each of a plurality of panelized modules of a panel including the plurality of panelized modules, wherein the panel is formed of a flexible circuit board such that each of the plurality of panelized modules defines a second circuit assembly;
mounting an imaging mechanism and a lighting mechanism onto each of the first circuit assemblies that are coupled to the plurality of panelized modules;
attaching an end cap over each of the first circuit assemblies that include the imaging mechanism and the lighting mechanism mounted thereon, the end cap is configured to house the imaging mechanism and the lighting mechanism;
filling the end cap with one or more materials to encapsulate the imaging mechanism and the lighting mechanism within the end cap; and
detaching the plurality of panelized modules from the panel to form a plurality of modular tip assemblies for attachment to a shaft of the plurality of medical devices.
